(19) United States
(12) Patent Application Publication
(10) Pub. No.: US 2021/0197183 A1
(43) Pub. Date: Jul. 1, 2021

(54) CHIRAL CATALYST AND HETEROGENEOUS CHIRAL CATALYST COMPRISING THE SAME

(71) Applicant: INDUSTRIAL TECHNOLOGY RESEARCH INSTITUTE, Hsinchu (TW)

(72) Inventors: Shih-Hsien LIU, Jhubei City (TW); Yi-Liang TSAI, Taichung City (TW); Chih-Lung CHIN, Hsinchu City (TW); Chien-Wen LIN, Taoyuan City (TW); Chao-Wu LIAW, Yunlin County (TW)

(73) Assignee: INDUSTRIAL TECHNOLOGY RESEARCH INSTITUTE, Hsinchu (TW)

(21) Appl. No.: 17/129,339

(22) Filed: Dec. 21, 2020

Related U.S. Application Data
(63) Continuation-in-part of application No. 16/730,345, filed on Dec. 30, 2019.

(30) Foreign Application Priority Data
Jul. 3, 2020 (TW) 109122521

(51) Int. Cl. B01J 31/02; B01J 21/08; C07F 7/18; C07C 29/143

(52) U.S. Cl. CPC B01J 31/0275; B01J 21/08; B01J 2531/002; C07C 29/143; B01J 2231/643; C07F 7/1804

(57) ABSTRACT

A chiral catalyst represented by formula (I) is provided. In formula (I), Z═Z₁ or Z₂, and the combination of Z₁ and Z₂ in formula (I) includes

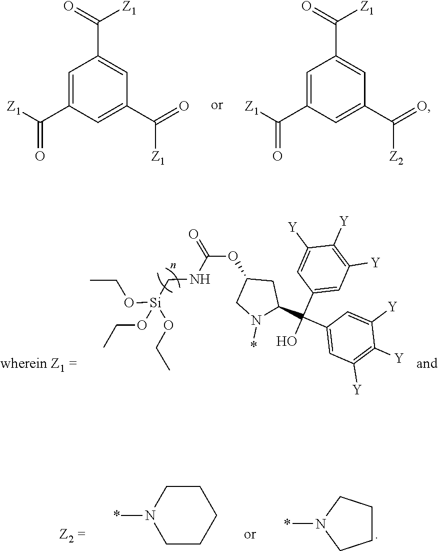

Y independently includes hydrogen, fluorine, trifluoromethyl, isopropyl, tert-butyl, CₘH₂ₘ₊₁ or OCₘH₂ₘ₊₁, m=1-10, and n=1-10. A heterogeneous chiral catalyst including the chiral catalyst is also provided.

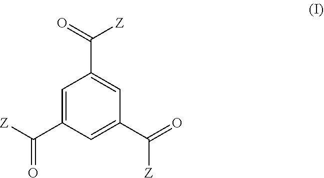

### CROSS REFERENCE TO RELATED APPLICATIONS

This application is a Continuation-In-Part of application Ser. No. 16/730,345, filed on Dec. 30, 2019, and claims priority of Taiwan Patent Application No. 109122521, filed on Jul. 3, 2020, the entirety of which is incorporated by reference herein.

### TECHNICAL FIELD

The present disclosure relates to a chiral catalyst for selective reduction of enantiomers, and a heterogeneous chiral catalyst including the chiral catalyst.

### BACKGROUND

Chiral catalysts' intermolecular forces and steric hindrances can induce reactions to form dextrorotatory molecules (prefixed with “(+)-”) or levorotatory molecules (prefixed with “(−)-”). Generally speaking, the criteria for success in the application of chiral catalysts to asymmetric synthesis relies on the high optical purity of the products, the recyclablility of the chiral catalysts, both dextrorotatory molecules and levorotatory molecules can be prepared separately, and a high conversion rate of the products.

However, in a homogeneous reaction, the chiral catalyst will eventually be mixed with the target products, increasing the difficulty and cost of recovery of the catalysts.

### SUMMARY

In accordance with one embodiment of the present disclosure, a chiral catalyst represented by formula (I) is provided.

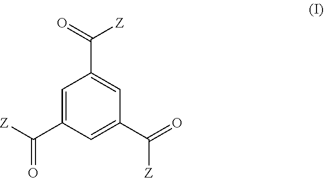

In formula (I), Z═Z₁ or Z₂, and the combination of Z₁ and Z₂ includes

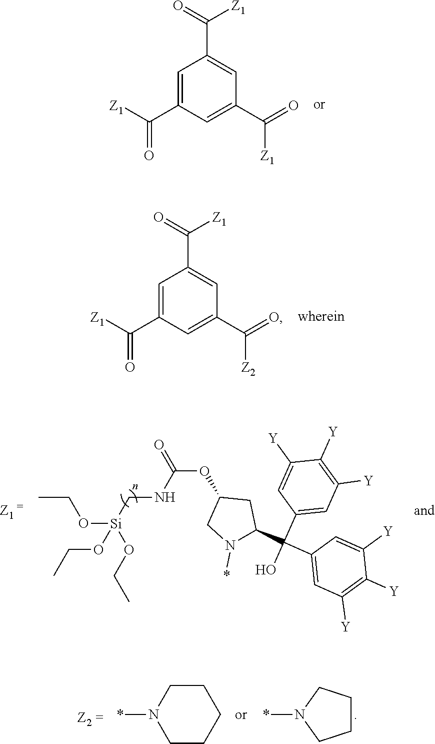

Y independently includes hydrogen, fluorine, trifluoromethyl, isopropyl, tert-butyl, CₘH₂ₘ₊₁ or OCₘH₂ₘ₊₁, m=1-10, and n=1-10.

In accordance with one embodiment of the present disclosure, a heterogeneous chiral catalyst is provided. The heterogeneous chiral catalyst includes the disclosed chiral catalyst and a substrate connected to the chiral catalyst.

A detailed description is given in the following embodiments.

### DETAILED DESCRIPTION

In accordance with one embodiment of the present disclosure, a chiral catalyst represented by formula (I) is provided.

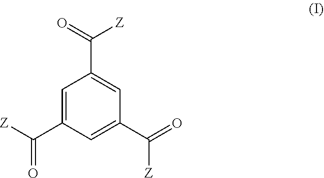

In formula (I), Z═Z₁ or Z₂, and the combination of Z₁ and Z₂ includes

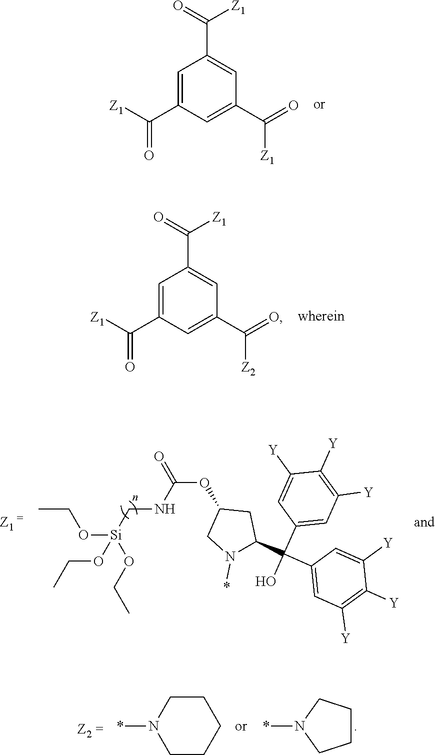

Y independently includes hydrogen, fluorine, trifluoromethyl, isopropyl, tert-butyl, CₘH₂ₘ₊₁ or OCₘH₂ₘ₊₁, m=1-10, and n=1-10.

In some embodiments, in formula (I), Y independently includes hydrogen, CH₃ or OCH₃, and n=3-8.

In some embodiments, the chiral catalyst is represented by formula (II) or (III):

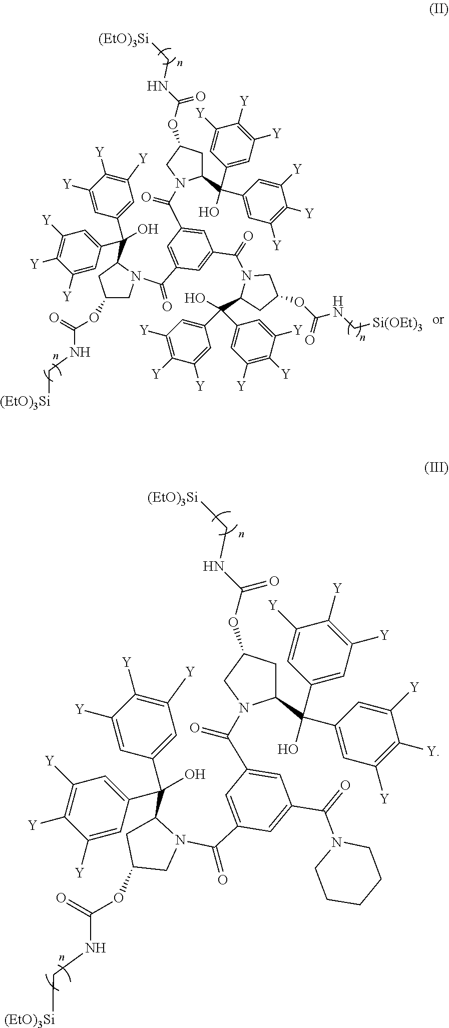

In formula (II) and (III), Y independently includes hydrogen, fluorine, trifluoromethyl, isopropyl, tert-butyl, CₘH₂ₘ₊₁ or OCₘH₂ₘ₊₁, m=1-10, and n=1-10.

In some embodiments, in formula (II) and (III), Y independently includes hydrogen, CH₃ or OCH₃, and n=3-8.

In some embodiments, the chiral catalyst may include the following compounds:

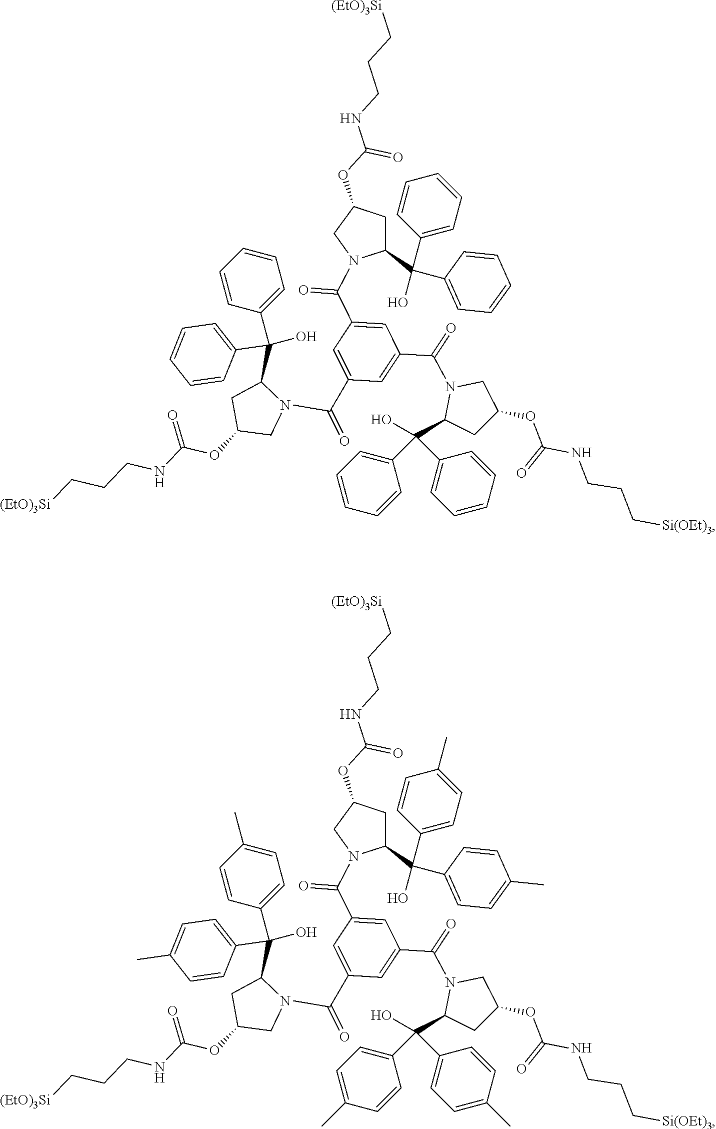

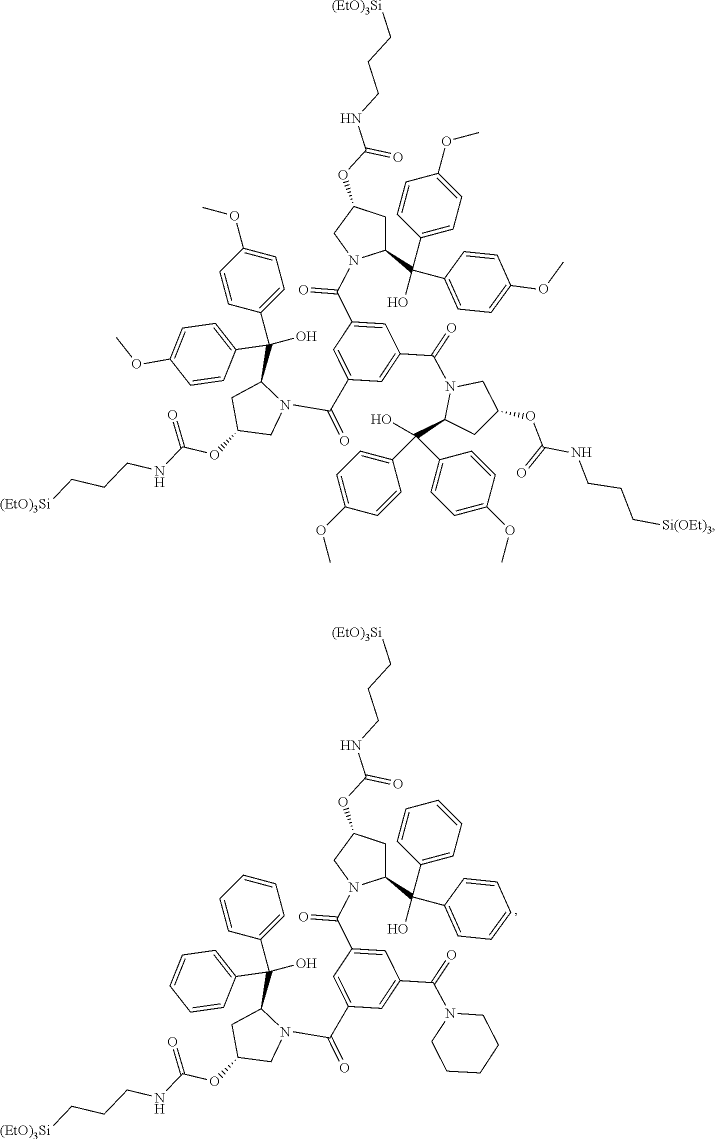

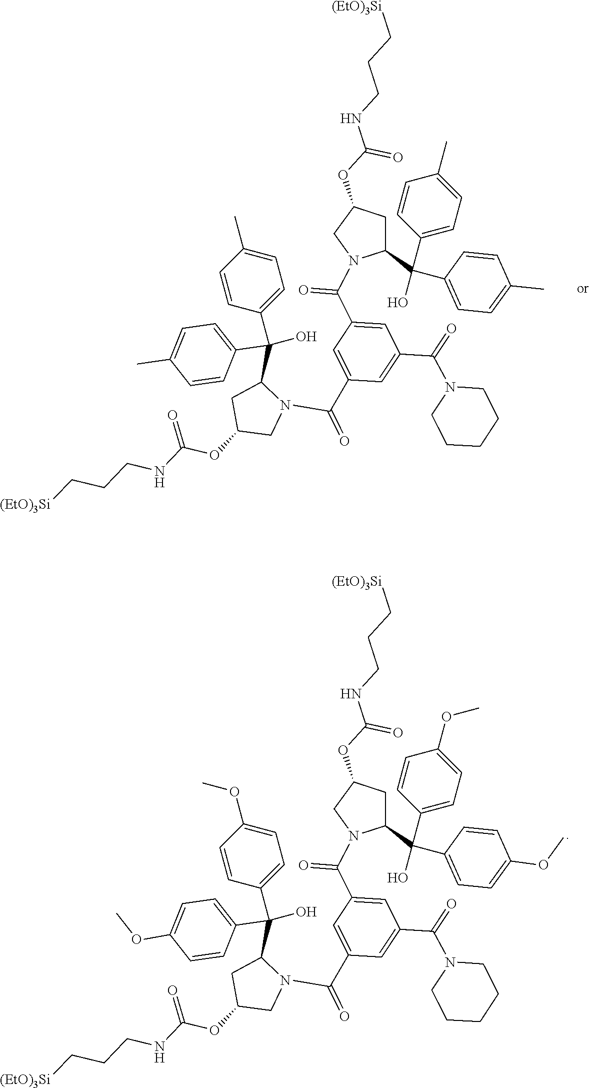

In accordance with one embodiment of the present disclosure, a heterogeneous chiral catalyst is provided. The heterogeneous chiral catalyst includes the disclosed chiral catalyst and a substrate connected to the chiral catalyst.

In accordance with one embodiment of the present disclosure, the heterogeneous chiral catalyst is represented by formula (IV):

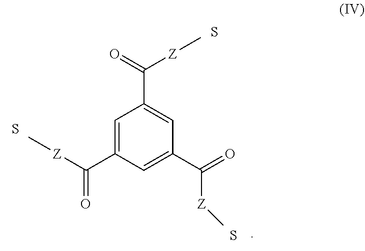

In formula (IV), S is the substrate, Z═Z₁ or Z₂, and the combination of Z₁ and Z₂ includes

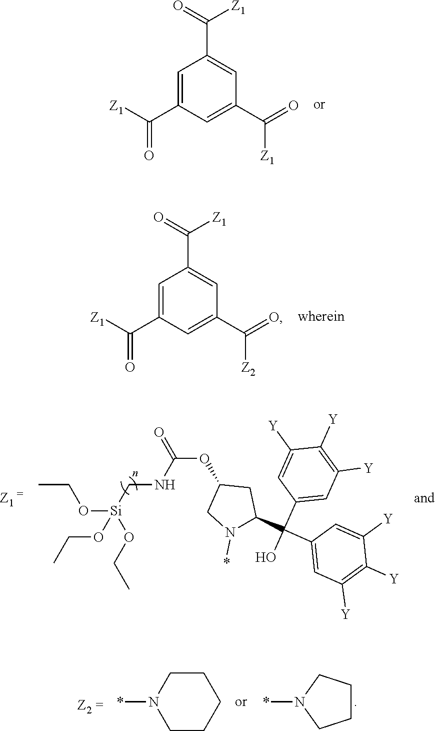

Y independently includes hydrogen, fluorine, trifluoromethyl, isopropyl, tert-butyl, CₘH₂ₘ₊₁ or OCₘH₂ₘ₊₁, m=1-10, and n=1-10.

In some embodiments, in formula (IV), Y independently includes hydrogen, CH₃ or OCH₃, and n=3-8.

In some embodiments, the heterogeneous chiral catalyst is represented by formula (V) or (VI), and S is the substrate:

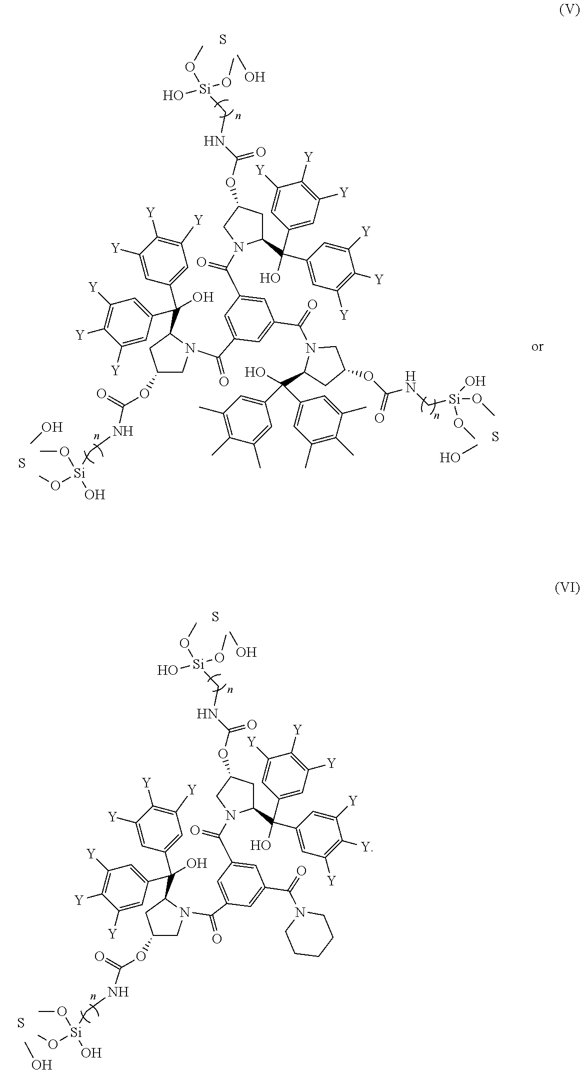

In formula (V) and (VI), Y independently includes hydrogen, fluorine, trifluoromethyl, isopropyl, tert-butyl, CₘH₂ₘ₊₁ or OCₘH₂ₘ₊₁, m=1-10, and n=1-10.

In some embodiments, in formula (V) and (VI), Y independently includes hydrogen, CH₃ or OCH₃, and n=3-8.

In some embodiments, the heterogeneous chiral catalyst may include the following compounds, and S is the substrate:

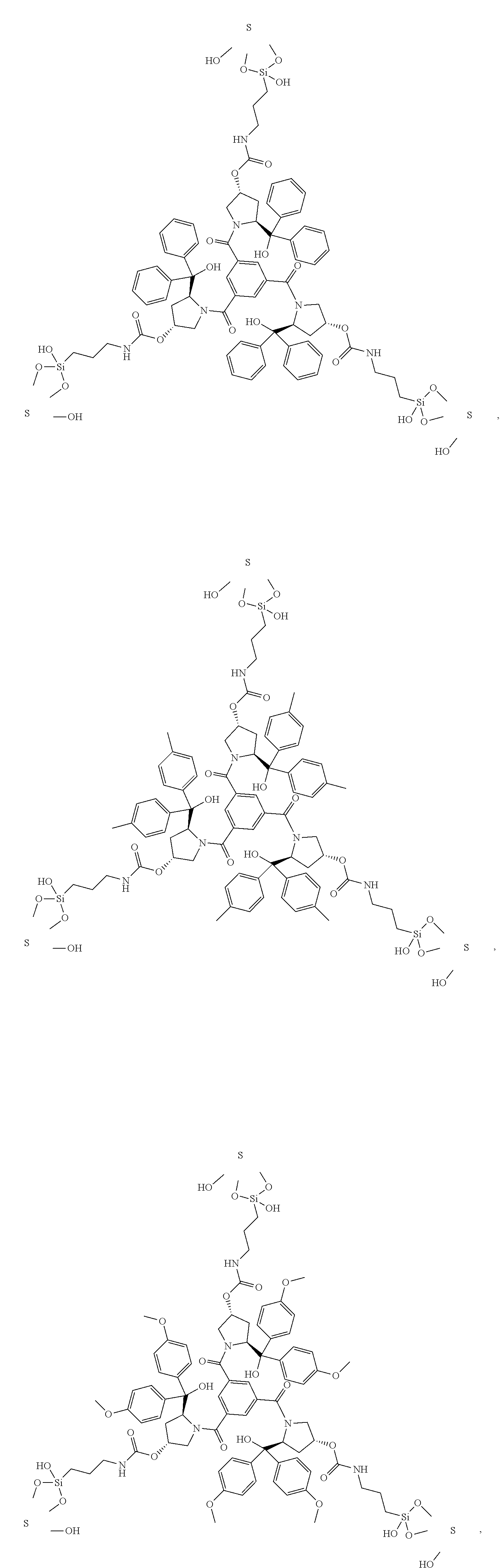

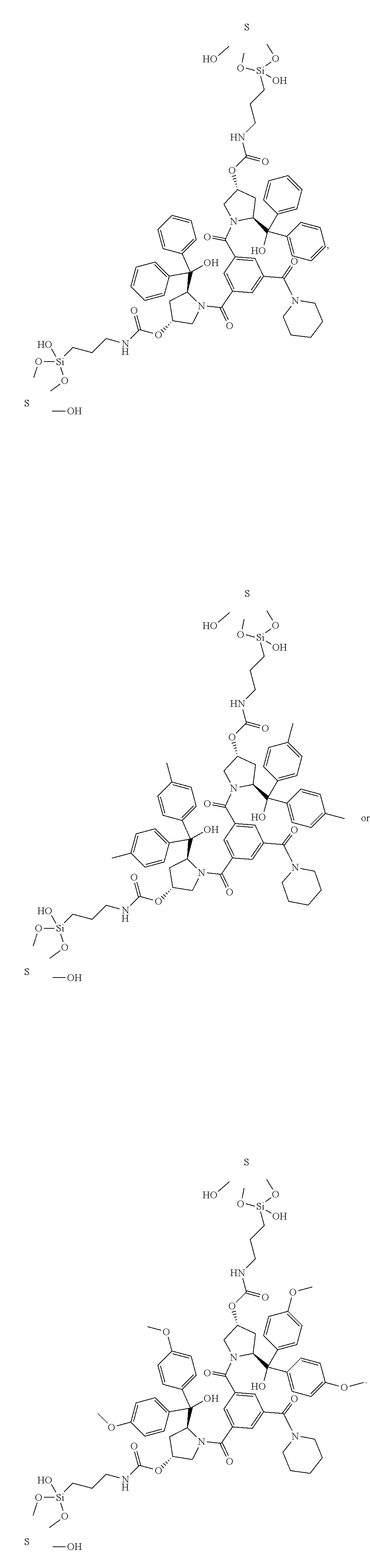

In some embodiments, in formula (IV), the substrate may include silicon oxide, titanium oxide, iron oxide, zinc oxide or aluminum oxide which are modified with a hydroxyl group on the surface thereof. In some embodiments, the substrate may include mesoporous material. In some embodiments, the specific surface area of the substrate is in a range from about 10 m²/g to about 1,000 m²/g. In some embodiments, the pore size of the substrate is in a range from about 2 nm to about 50 nm. In some embodiments, the hydroxyl group of S is connected to the Si(OEt)₃ group of Z1. In some embodiments, a silicon-oxygen bond is formed between the substrate and Z1. In some embodiments, the average particle size of the substrate is in a range from about 5 μm to about 500 μm or from about 30 μm to about 300 μm.

In some embodiments of the present disclosure, the chiral catalyst molecule having two or three side chains with silanization further forms a covalent bond with SiO₂ and is fixed on the surface of SiO₂. When solvent and ketone compounds (reactants) flow through, the reactants react with the chiral catalyst on SiO₂ to proceed to a catalytic reaction. After the reaction is complete, the synthesized chiral alcohol compounds are removed with the flow of the solvent, which helps to separate the product from the chiral catalyst, facilitates recycling and reuse, decreases the difficulty of the recovery of the chiral catalyst from a homogeneous reaction, and improves the reuse rate of the chiral catalyst. The selective reduction method involving this chiral catalyst can effectively increase the optical purity and conversion rate of the product. In addition, the present disclosure can implement a continuous reduction reaction and synthesize chiral alcohol compounds in a more economical and efficient manner.

### Example 1

Preparation of the Chiral Catalyst (1)

Step 1

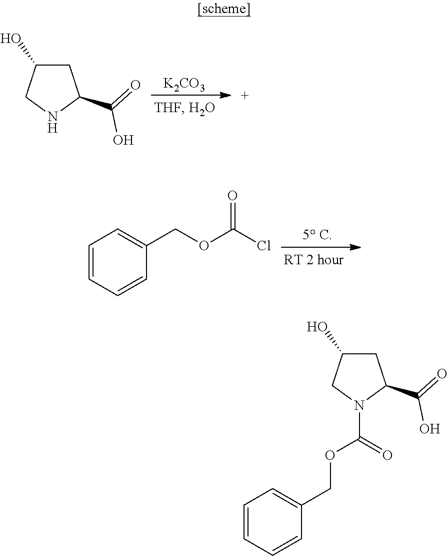

[symbol] Reagent

[symbol]

| | | molecular | | | | molar |
|---|---|---|---|---|---|---|
| item | reactant | weight | amount | | mmole | ratio |
| 1 | trans-4-hydroxy- | 131.13 | 30 | g | 228.78 | 1 |
| | L-proline | | | | | |
| 2 | potassium carbonate | 138.21 | 39.52 | g | 285.97 | 1.25 |
| 3 | tetrahydrofuran/ | — | 120/160 | mL | — | — |
| | water | | | | | |
| 4 | benzyl | 170.59 | 35.8/120 | mL | 251.66 | 1.1 |
| | chloroformate/water | | | | | |

[symbol] Synthesis Steps

[symbol]

First, a 1-L double-necked reaction flask was provided, after being purged with nitrogen, items 1-3 were added to the flask and stirred until completely dissolved. Next, the reaction flask was placed in an ice bath with conduction of nitrogen to cool down, and the temperature in the ice bath was maintained at 0-5° C. Next, item 4 solvent was slowly dripped into the reaction flask. After the dripping finished, the temperature of the reaction solution was allowed to return to room temperature while stirring for 2 hours. Next, an HPLC measurement (hexane (Hex): isopropanol (IPA)=4:1 and 0.1% trifluoroacetic acid (TFA)) with flow rate of 0.5 mL/min was performed, and the retention time of the product appeared at 14.92 min. After the reaction was complete, 300 mL of H₂O was added, and extracted with 100 mL of ethanolamine (EA) each time and repeated 4 times (total 400 mL of EA). The EA layer was inspected by HPLC and no product was detected in the EA layer. Next, the aqueous layer was acidified with a 3M HCl aqueous solution to pH=2, and then extracted with 100 mL of EA each time and repeated 4 times (total 400 mL of EA). The EA layer was dehydrated with anhydrous magnesium sulfate and filtered, and then the solvent was removed at 40° C. using a rotary concentrator. Next, a vacuum drying step was performed at room temperature for 12 hours and 54 g of transparent liquid was obtained. The yield was 89.0%, and an HPLC measurement (Hex:IPA=4:1 and 0.1% TFA) with flow rate of 0.5 mL/min was performed, and the product appeared at 14.70 min [REGIS (S,S) Whelk-O1 5 μm, 4.6×150 mm]. The purity thereof was 99.2%. The target product was measured by NMR. Data are as follows: ¹H NMR (400 MHz, DMSOd₆): δ 12.63 (s, 1H, COOH), 7.36-7.28 (m, 5H), 5.10-5.00 (m, 3H, OH, PhCH₂O), 4.30-4.19 (m, 2H), 3.50-3.36 (m, 2H), 2.21-2.11 (m, 1H), 1.99-1.81 (m, 1H).

Step 2

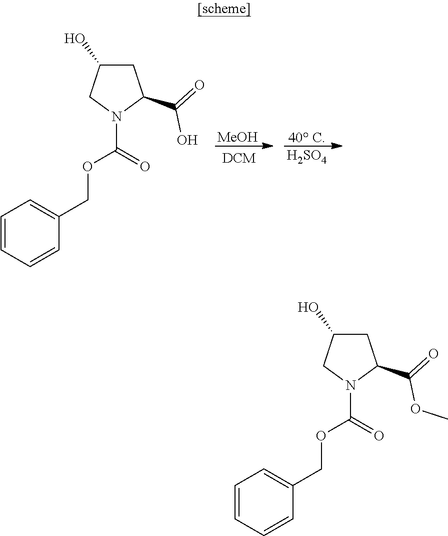 [SMILES: "MeOH" ; O=C(O)[C@@H]1C[C@@H](O)CN1C(=O)OCc1ccccc1 ; "H2SO4" ; "40° C." ; COC(=O)[C@@H]1C[C@@H](O)CN1C(=O)OCc1ccccc1]

[symbol] Reagent

[symbol]

| | | molecular | | | | molar |
|---|---|---|---|---|---|---|
| item | reactant | weight | amount | | mmole | ratio |
| 1 | product of Step 1/ | 265.26 | 54 g/75 mL | | 203.57 | 1 |
| | methanol | | | | | |
| 2 | methanol/ | — | 50/250 | mL | — | — |
| | dichloromethane | | | | | |
| 3 | sulfuric acid, | 98.08 | 1.5 | mL | 28.11 | 0.15 |
| | D = 1.84 | | | | | |

| | | | | | |
|---|---|---|---|---|---|
| 4 | potassium | 138.21 | 8.4 g/200 mL | 61.07 | 0.3 |
| | carbonate/water | | | | |

[symbol] Synthesis Steps

[symbol]

First, a 500-mL double-necked reaction flask was provided, after being purged with nitrogen, items 1-3 were added to the flask and stirred until completely dissolved. Next, the reaction flask was placed in an oil bath with conduction of nitrogen to cool down, and the temperature in the oil bath was maintained at 40-50° C. Next, the reaction was tracked by HPLC (Hex:IPA=4:1 and 0.1% TFA) with flow rate of 1.0 mL/min, and the product appeared at 11.53 min. The reaction was complete after 12 hours. After the reaction was complete, the solution was concentrated to 20-30 mL. Item 4 aqueous solution was added and extracted with 100 mL of EA each time and repeated 3 times (total 300 mL of EA). The EA layer was dehydrated with anhydrous magnesium sulfate and filtered, and then the solvent was removed at 50° C. using a rotary concentrator. Next, a vacuum drying step was performed at room temperature for 12 hours and 57.8 g of light-yellow transparent liquid was obtained. The yield was 79.5%, and an HPLC measurement (Hex:IPA=4:1 and 0.1% TFA) with flow rate of 1.0 mL/min was performed, and the product appeared at 11.507 min [REGIS (S,S) Whelk-O1 5 μm, 4.6×150 mm]. The purity thereof was 98.0%. The target product was measured by NMR. Data are as follows: H NMR (400 MHz, CDCl₃): δ 7.32-7.24 (m, 5H), 5.18-4.96 (m, 2H, PhCH2O), 4.51-4.44 (m, 2H), 3.72-3.59 (m, 3H), 3.54-3.52 (m, 2H), 2.32-2.23 (m, 1H), 2.07-1.80 (m, 1H).

Step 3

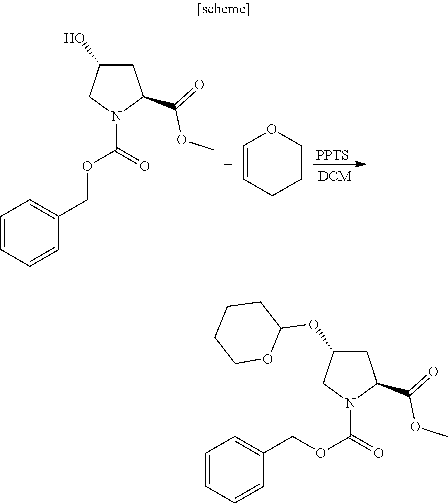

[symbol] Reagent

[symbol]

| | | molecular | | | | molar |
|---|---|---|---|---|---|---|
| item | reactant | weight | amount | | mmole | ratio |
| 1 | product of Step 2 | 279.29 | 57.9 | g | 207.3 | 1 |
| 2 | 3,4-dihydro-2H- | 84.12 | 26.15 | g | 310.97 | 1.5 |
| | pyran | | | | | |
| 3 | pyridinium p- | 251.3 | 0.52 | | 2.078 | 0.01 |
| | toluenesulfonate | | | | | |
| 4 | dichloromethane | — | 600 | mL | — | — |

[symbol] Synthesis Steps

[symbol]

First, a 1-L double-necked reaction flask was provided, after being purged with nitrogen, items 1-4 were added to the flask and stirred until completely dissolved. Next, the reaction flask was placed in an oil bath with conduction of nitrogen to cool down, and the temperature in the oil bath was maintained at 40° C. Next, the reaction was tracked by HPLC (Hex:IPA=4:1 and 0.1% TFA) with flow rate of 1.0 mL/min, and the product appeared at 18.613 min. The reaction was complete after 24 hours. After the reaction was complete, 300 mL of H₂O was added and extracted with DCM. The DCM layer was dehydrated with anhydrous magnesium sulfate and filtered, and then the solvent was removed at 40° C. using a rotary concentrator. Next, a vacuum drying step was performed at room temperature for 12 hours and 74.7 g of transparent liquid was obtained. The yield was 99.2%, and an HPLC measurement (Hex:IPA=4:1 and 0.1% TFA) with flow rate of 1.0 mL/min was performed, and the product appeared at 17.52 min [REGIS (S,S) Whelk-O1 5 μm, 4.6×150 mm]. The purity thereof was 99.2%. The target product was measured by NMR. Data are as follows: H NMR (400 MHz, CDCl₃): δ 7.32-7.26 (m, 5H), 5.19-4.98 (m, 2H, PhCH2O), 4.63-4.58 (m, 1H), 4.50-4.37 (m, 2H), 3.83-3.63 (m, 3H+2H), 3.54-3.41 (m, 2H), 2.45-2.29 (m, 1H), 2.14-2.01 (m, 1H), 1.75-1.62 (m, 2H), 1.58-1.39 (m, 4H).

Step 4

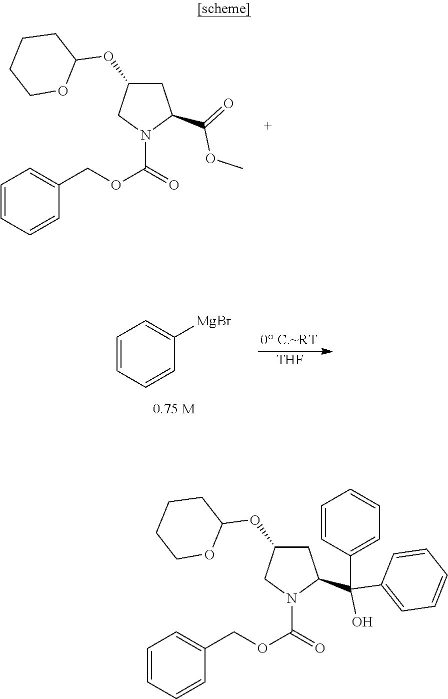

[symbol] Reagent

[symbol]

| | | molecular | | | molar |
|---|---|---|---|---|---|
| item | reactant | weight | amount | mmole | ratio |
| 1 | product of Step 3/ | 363.40 | 20 g/200 mL | 55.03 | 1 |
| | tetrahydrofuran | | | | |
| 2 | bromobenzene/ | 157.01 | 34.56 g/70 mL | 220.14 | 4 |
| | tetrahydrofuran | | | | |
| 3 | magnesium, | 24.31 | 5.62 g/10 mL | 231.12 | 4.2 |
| | turnings/ | | | | |
| | tetrahydrofuran | | | | |
| 4 | tetrahydrofuran, | — | 100 mL | — | — |
| | dry | | | | |

[symbol] Synthesis Steps

[symbol]

First, a 250-mL double-necked reaction flask was provided, after being purged with nitrogen, item 3 was added to the flask and stirred. After about 10-15 mL of item 2 was added to the reaction flask dropwise, the reaction was started by heating with a blower and boiled. After item 2 was slowly added to the system dropwise, the reaction flask was placed in an oil bath, and the temperature of the oil bath was maintained at 50-60° C. Another 500-mL double-necked reaction flask was provided, purged with nitrogen, and then item 1 was added to the flask and stirred to cool down to 0° C. to −5° C. Next, the reaction solution obtained by the above steps was placed in a feeding funnel and slowly dripped into the reaction solution, and the internal temperature was maintained at 0-10° C. After the dripping finished, the solution was heated to 40° C. and reacted for about 2 hours. Next, the reaction was tracked by HPLC (Hex:IPA=4:1 and 0.1% TFA) with flow rate of 1.0 mL/min, and the product appeared at 11.98 min. After the reaction was complete, 3M HCl aqueous solution was added to neutralize to pH=6-8, and extracted with 100 mL of EA each time and repeated 3 times (total 300 mL of EA). The EA layer was dehydrated with anhydrous magnesium sulfate and filtered, and then the solvent was removed at 50° C. using a rotary concentrator. Next, a column (3 cm in diameter) packing 40 cm (SILICYCLE Silica gel 70-230 mesh, pH=7) was provided. After impurities were eluted with EA:Hex=1:10, varied the ratio of EA:Hex to 1:4, and the product was eluted. About 30-50 mL of solvent was removed at 40° C. using a rotary concentrator. Solid was precipitated and filtered. Next, a vacuum drying step was performed at 60° C. for 12 hours, and 16.7 g of white solid was obtained. The yield was 62.3%, and an HPLC measurement (Hex:IPA=4:1 and 0.1% TFA) with flow rate of 1.0 mL/min was performed, and the product appeared at 12.053 min [REGIS (S,S) Whelk-O1 5 μm, 4.6×150 mm]. The purity thereof was 98.5%. The target product was measured by NMR. Data are as follows: H NMR (400 MHz, CDCl3): δ 7.37-7.24 (m, 15H), 5.10-4.99 (m, 2H, PhCH2O), 4.40-4.36 (d, 1H), 3.71-3.66 (m, 2H+1H), 3.39-3.32 (m, 1H), 2.23-2.09 (m, 2H), 1.70-1.69 (m, 1H), 1.62-1.24 (m, 8H).

Step 5

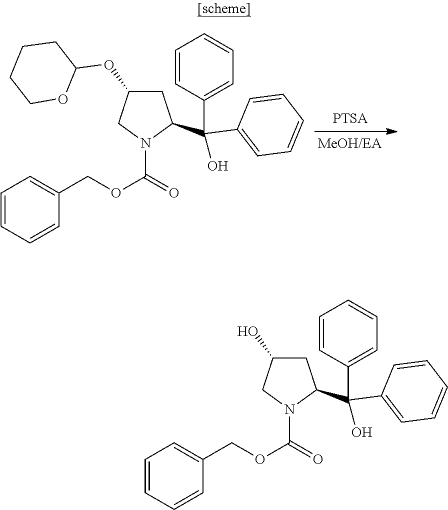 [SMILES: O=C(OCc1ccccc1)N1C[C@H](OC2CCCCO2)C[C@H]1C(O)(c1ccccc1)c1ccccc1 ; O=C(OCc1ccccc1)N1C[C@H](O)C[C@H]1C(O)(c1ccccc1)c1ccccc1]

[symbol] Reagent

[symbol]

| | | molecular | | | | molar |
|---|---|---|---|---|---|---|
| item | reactant | weight | amount | | mmole | ratio |
| 1 | product of Step 4 | 487.59 | 10 | g | 20.51 | 1 |
| 2 | p-toluenesulfonic | 190.22 | 0.04 | g | 0.205 | 0.01 |
| | acid | | | | | |
| 3 | ethyl acetate | — | 50 | mL | — | — |
| 4 | methanol | — | 50 | mL | — | — |

[symbol] Synthesis Steps

[symbol]

First, a 250-mL double-necked reaction flask was provided, after being purged with nitrogen, items 1-4 were added to the flask while stirring and heated to an internal temperature of 50-60° C. Next, the reaction was tracked by HPLC (Hex:IPA=4:1 and 0.1% TFA) with flow rate of 1.0 mL/min, and the product appeared at 8.974 min. The reaction was complete after about 4 hours. After the reaction was complete, EA was used for extraction. Each extraction was performed with 100 mL of EA and repeated 3 times (total 300 mL of EA). The EA layer was dehydrated with anhydrous magnesium sulfate and filtered, and then the solvent was removed at 50° C. using a rotary concentrator. Next, a vacuum drying step was performed at 60° C. for 12 hours and 8.3 g of white solid was obtained. The yield was 99.0%, and an HPLC measurement (Hex:IPA=4:1 and 0.1% TFA) with flow rate of 1.0 mL/min was performed, and the product appeared at 8.947 min [REGIS (S,S) Whelk-O1 5 μm, 4.6×150 mm]. The purity thereof was 88.0%. Without purification, the next step was performed. The target product was measured by NMR. Data are as follows: H NMR (400 MHz, CDCl₃): δ 7.41-7.27 (m, 15H), 5.18-5.13 (m, 2H, PhCH2O), 4.97 (s, 1H), 3.93 (s, 1H), 3.62-3.59 (d, 1H), 3.08-3.06 (d, 1H), 2.20-1.98 (m, 2H), 1.62-1.60 (m, 2H).

Step 6

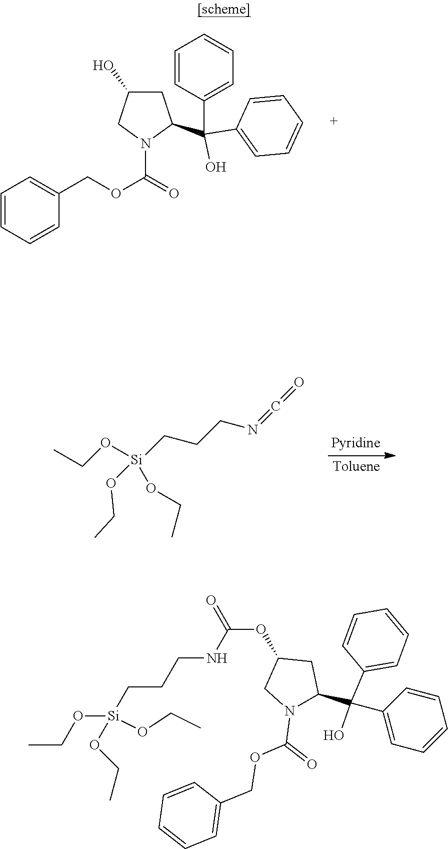 [SMILES: O=C(OCc1ccccc1)N1C[C@H](O)C[C@H]1C(O)(c1ccccc1)c1ccccc1 ; CCO[Si](CCCN=C=O)(OCC)OCC ; CCO[Si](CCCNC(=O)O[C@@H]1C[C@@H](C(O)(c2ccccc2)c2ccccc2)N(C(=O)OCc2ccccc2)C1)(OCC)OCC]

[symbol] Reagent

[symbol]

| | | molecular | | | | molar |
|---|---|---|---|---|---|---|
| item | reactant | weight | amount | | mmole | ratio |
| 1 | product of Step 5 | 403.47 | 4 | g | 9.88 | 1 |
| 2 | 3-(triethoxy- | 247.36 | 4.9 | g | 19.76 | 2 |
| | silyl)propyl | | | | | |
| | isocyanate | | | | | |
| 3 | pyridine | 79.1 | 2.34 | g | 29.64 | 3 |
| 4 | toluene | — | 40 | mL | — | — |

[symbol] Synthesis Steps

[symbol]

First, a 100-mL double-necked reaction flask was provided, after being purged with nitrogen, items 1-4 were added to the flask while stirring and heated to thermal reflux. Next, the reaction was tracked by HPLC (Hex:IPA=4:1 and 0.1% TFA) with flow rate of 1.0 mL/min, and the product appeared at 13.24 min. The reaction was complete after about 3 days. After the reaction was complete, EA was used for extraction. Each extraction was performed with 50 mL of EA and repeated 3 times (total 150 mL of EA). The EA layer was dehydrated with anhydrous magnesium sulfate and filtered, and then the solvent was removed at 50° C. using a rotary concentrator. Next, a column (3 cm in diameter) packing 40 cm (SILICYCLE Silica gel 70-230 mesh, pH=7) was provided. After impurities were eluted with EA:Hex=1:10, varied the ratio of EA:Hex to 1:4, and the product was eluted. About 30-50 mL of solvent was removed at 40° C. using a rotary concentrator. Solid was precipitated and filtered. Next, a vacuum drying step was performed at room temperature for 12 hours, and 2.75 g of transparent liquid was obtained. The yield was 45.5%, and an HPLC measurement (Hex:IPA=4:1 and 0.1% TFA) with flow rate of 1.0 mL/min was performed, and the product appeared at 13.2 min [REGIS (S,S) Whelk-O1 5 μm, 4.6×150 mm]. The purity thereof was 94.2%. The target product was measured by NMR. Data are as follows: ¹H NMR (400 MHz, CDCl3): δ 7.39-7.18 (m, 15H), 5.11-5.03 (m, 2H, PhCH2O), 4.87-4.85 (m, 2H), 4.12-4.07 (m, 1H), 3.81-3.78 (m, 6H), 3.71-3.64 (m, 1H), 3.11-3.05 (m, 2H), 2.25-2.10 (m, 2H), 1.60-1.52 (m, 2H), 1.25-1.16 (m, 9H), 0.61-0.51 (m, 2H).

Step 7

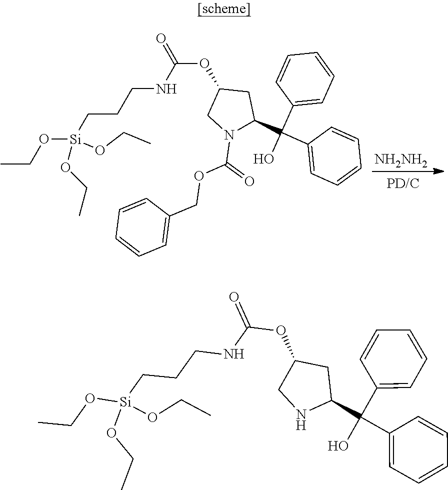

[symbol] Reagent

[symbol]

| | | molecular | | | | molar |
|---|---|---|---|---|---|---|
| item | reactant | weight | amount | | mmole | ratio |
| 1 | product of Step 6 | 650.83 | 2 | g | 3.06 | 1 |
| 2 | 5% palladium on | — | 0.2 | g | — | — |
| | carbon | | | | | |
| 3 | hydrazine | 50.06 | 0.23 | g | 4.6 | 1.5 |
| | monohydrate | | | | | |
| 4 | methanol | — | 20 | mL | — | — |

[symbol] Synthesis Steps

[symbol]

First, a 100-mL double-necked reaction flask was provided, purged with nitrogen, and then items 1-4 were added to the flask while stirring and heated to an internal temperature of 50-60° C. Next, the reaction was tracked by HPLC (Hex:IPA=4:1 and 0.1% TFA) with flow rate of 1.0 mL/min, and the product appeared at 4.787 min. The reaction was complete after about 3 hours. Next, the solvent was removed at 50° C. using a rotary concentrator. Next, a column (3 cm in diameter) packing 20 cm (SILICYCLE Silica gel 70-230 mesh, pH=7) was provided using an eluent (EA:Hex=1:6). After impurities were washed out, the eluent (EA:Hex=1:1) was used to elute the product. The product was then concentrated and dried. After a vacuum drying step was performed at room temperature for 12 hours, 0.6 g of transparent liquid was obtained. The yield was 38.0%, and an HPLC measurement (Hex:IPA=4:1 and 0.1% TFA) with flow rate of 0.5 mL/min was performed, and the product appeared at 10.2 min [REGIS (S,S) Whelk-O1 5 μm, 4.6×150 mm]. The purity thereof was 93.0%. The target product was measured by NMR. Data are as follows: H NMR (400 MHz, CDCl₃): δ 7.58-7.53 (m, 2H), 7.44-7.42 (m, 2H), 7.30-7.11 (m, 6H), 5.06 (s, 1H), 4.90 (s, 1H), 4.51-4.47 (m, 1H), 3.82-3.77 (m, 6H), 3.26-3.22 (m, 2H), 3.15-3.04 (m, 2H), 1.63-1.51 (m, 4H), 1.22-1.19 (m, 9H), 0.62-0.58 (m, 2H).

Step 8

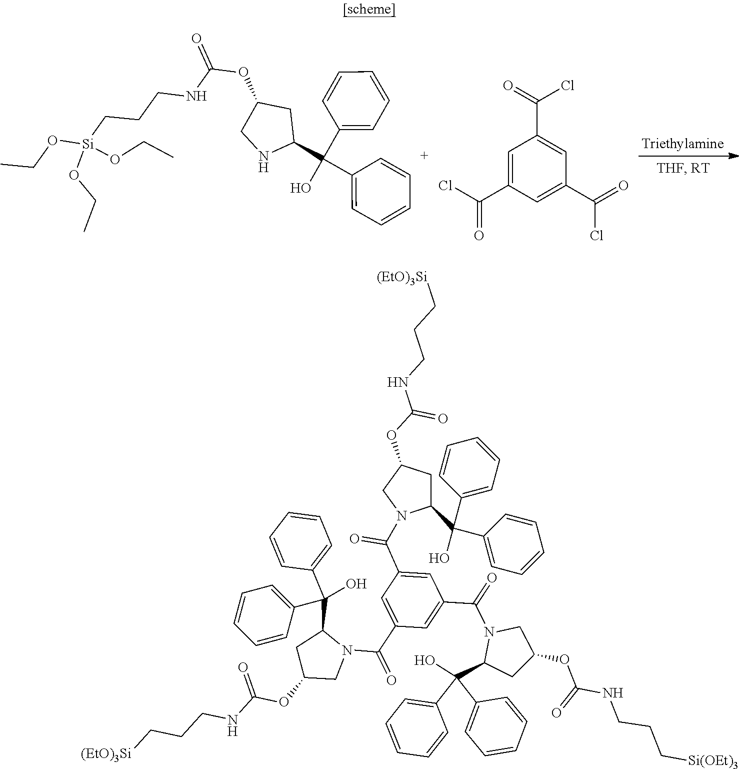

[symbol]

Reagent

[symbol]

| | | molecular | | | | molar |
|---|---|---|---|---|---|---|
| item | reactant | weight | amount | | mmole | ratio |
| 1 | product of Step 7 | 516.70 | 0.53 | g | 1.025 | 3.3 |
| 2 | 1,3,5-benzene- | 265.47 | 0.082 | g | 0.310 | 1 |
| | tricarbonyl | | | | | |
| | trichloride, | | | | | |
| | D = 1.487 | | | | | |
| 3 | tetrahydrofuran | — | 20 | mL | — | — |

| | | | | | |
|---|---|---|---|---|---|
| 4 | triethylamine, | 101.19 | 0.93 g/1.3 mL | 9.225 | 9 |
| | D = 0.726 | | | | |

[symbol] Synthesis Steps

[symbol]

First, a 100-mL double-necked reaction flask was provided, purged with nitrogen, and then items 1-3 were added to the flask with stirring. After item 4 was slowly dripped into the above reaction solution, solid salts were precipitated. The reaction was complete after about 24 hours. After the reaction was complete, EA was used for extraction. Each extraction was performed with 100 mL of EA and repeated 3 times (total 300 mL of EA). The EA layer was dehydrated with anhydrous magnesium sulfate and filtered, and then the solvent was removed at 35° C. using a rotary concentrator. The filtrate was dissolved in 10 mL of acetone and recrystallized using hexane. The product was filtered using the FP-450 filter paper (Life Sciences). Next, a vacuum drying step was performed at 40° C. for 12 hours, and 0.4 g of white solid was obtained. The yield was 77.6%, and an HPLC measurement (Hex:IPA=4:1 and 0.1% TFA) with flow rate of 0.5 mL/min was performed, and the product appeared at 5.427 min [REGIS (S,S) Whelk-O1 5 μm, 4.6×150 mm]. The purity thereof was 98.9%, and the signal that appeared within the first 5 minutes was solvent EA.

According to HPLC measurement, the reaction in Step 8 was completed, and the purity of the product was 98.9%. The target product was measured by NMR. Data are as follows: H NMR (400 MHz, CDCl₃): δ 7.59-7.52 (m, 2H), 7.45-7.41 (m, 2H), 7.35-7.10 (m, 9H), 5.05 (s, 1H), 4.94 (s, 1H), 4.50-4.45 (m, 1H), 3.80-3.75 (m, 6H), 3.27-3.30 (m, 2H), 3.18-2.93 (m, 2H), 1.64-1.51 (m, 4H), 1.23-1.18 (m, 9H), 0.60-0.57 (m, 2H). The target product was measured by mass spectrometry. Data are as follow: HRESI: Impact HD Q-TOF mass spectrometer (Bruker, Germany), calcd for C₉H₁₂₀N₆O₂₁Si₃=1705.78, found [M+Na]⁺=1728.75, Na=22.98.

### Example 2

Preparation of the Heterogeneous Chiral Catalyst

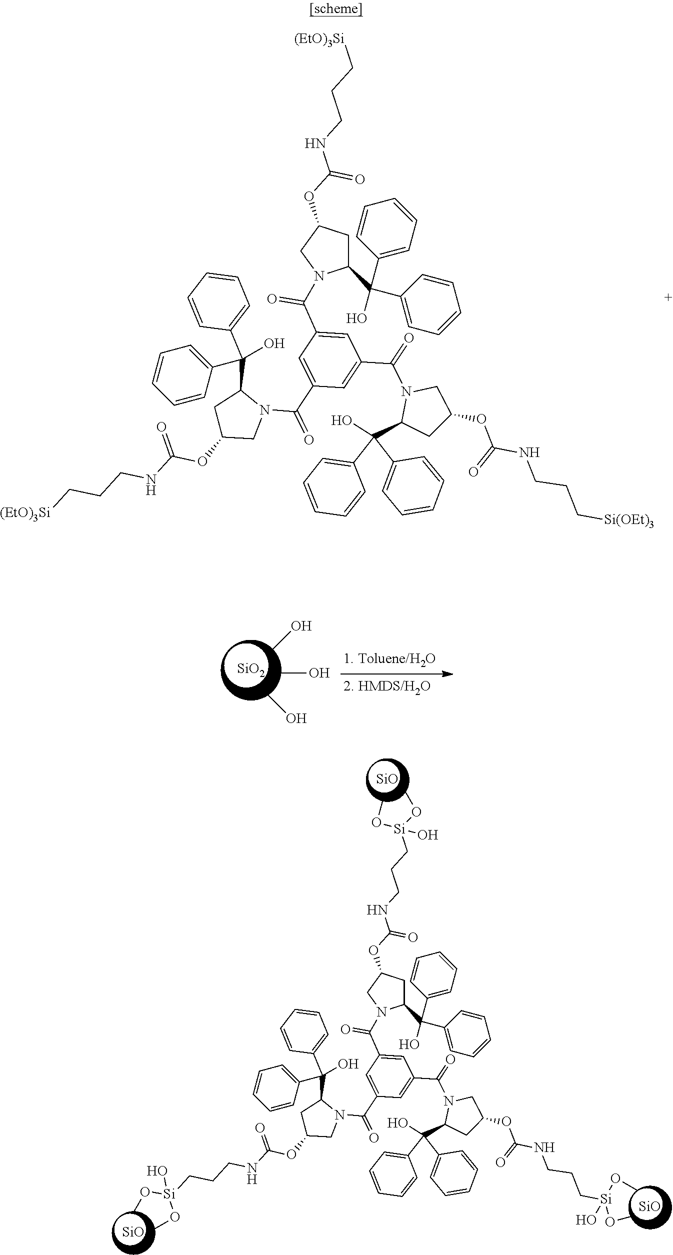

[symbol] Reagent

[symbol]

| | | molecular | | | | molar |
|---|---|---|---|---|---|---|
| item | reactant | weight | amount | | mmole | ratio |
| 1 | product of Step 8 | 1705.78 | 0.6 | g | — | 1 |
| 2 | surface-modified SiO₂ | — | 1.8 | g | — | 3 |
| | (heterogeneous chiral | | | | | |
| | catalyst (IV) G60, | | | | | |
| | purchased from Silicycle | | | | | |
| | company, model: G60, | | | | | |
| | average particle size: | | | | | |
| | about 60-200 μm, | | | | | |
| | pH: about 7) | | | | | |
| | (heterogeneous chiral | | | | | |
| | catalyst (V) SBA-15, | | | | | |
| | purchased from Aldrich | | | | | |
| | company, model: | | | | | |
| | mesoporous SBA-15, | | | | | |
| | specific surface area: | | | | | |
| | about 750 m²/g, average | | | | | |
| | particle size: less than | | | | | |
| | 150 μm, average pore size: | | | | | |
| | about 6 nm, pore capacity: | | | | | |
| | about 0.5-0.7 cm³/g) | | | | | |
| 3 | toluene | — | 24 | mL | — | — |
| 4 | DI water | — | 2.4 | μL | — | — |
| 5 | toluene | — | 24 | mL | — | — |
| 6 | DI water | — | 2.4 | μL | — | — |
| 7 | hexamethyldisilazane | 161.40 | 2.4 | g | | 4 |
| | (HMDS) | | | | | |

[symbol] Synthesis Steps

[symbol]

Heterogeneous Chiral Catalyst (IV)

First, a 100-mL double-necked reaction flask was provided, purged with nitrogen, and then items 1-4 were added to the flask while stirring and heated to 80° C. The reaction was complete after about 24 hours. The product was filtered using the FP-450 filter paper (Life Sciences). The solids were washed using a continuous extraction apparatus. The solvents used were methanol, acetone and dichloromethane. After cleaning, the product was filtered using the FP-450 filter paper (Life Sciences). A vacuum drying step was performed at 40° C. for 12 hours, and gray solids of 2.28 g were obtained. A 100-mL double-necked reaction flask was provided, purged with nitrogen, and the product obtained from the above step and items 5-7 were added to the flask while stirring and heated to 80° C. The reaction was complete after about 24 hours. The product was filtered using the FP-450 filter paper (Life Sciences). The solids were washed using a continuous extraction apparatus. The solvents used were methanol, acetone and dichloromethane. After washing, the product was filtered using the FP-450 filter paper (Life Sciences). A vacuum drying step was performed at 40° C. for 12 hours, and gray solids of 2.20 g were obtained. The target product was then measured by IR.

Heterogeneous Chiral Catalyst (V)

First, a 100-mL double-necked reaction flask was provided, purged with nitrogen, and then items 1-4 were added to the flask while stirring and heated to 80° C. The reaction was complete after about 24 hours. The product was filtered using the FP-450 filter paper (Life Sciences). The solids were washed using a continuous extraction apparatus. The solvents used were methanol, acetone and dichloromethane. After cleaning, the product was filtered using the FP-450 filter paper (Life Sciences). A vacuum drying step was performed at 40° C. for 12 hours, and gray solids of 2.13 g were obtained. A 100-mL double-necked reaction flask was provided, purged with nitrogen, and the product obtained from the above step and items 5-7 were added to the flask while stirring and heated to 80° C. The reaction was complete after about 24 hours. The product was filtered using the FP-450 filter paper (Life Sciences). The solids were washed using a continuous extraction apparatus. The solvents used were methanol, acetone and dichloromethane. After washing, the product was filtered using the FP-450 filter paper (Life Sciences). A vacuum drying step was performed at 40° C. for 12 hours, and gray solids of 2.11 g were obtained. The target product was then measured by IR.

The Results of IR Measurement

Heterogeneous chiral catalyst (IV): —CH₂ (2928 nm, 2854 nm, 1456 nm), —CONH (1645 nm), 3°-OH (1180-1250 nm), -Ph (702-754 nm).

Heterogeneous chiral catalyst (V): —CH₂ (2927 nm, 2857 nm, 1449 nm), —CONH (1643 nm), 3°-OH (1162-1245 nm), -Ph (702-753 nm).

### Example 3

Chiral Catalysts for Selective Reduction Reaction

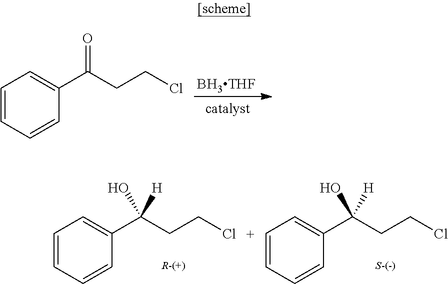

In this example, chiral catalyst (I) and catalysts (II) and (III) were provided to perform the selective reduction reaction.

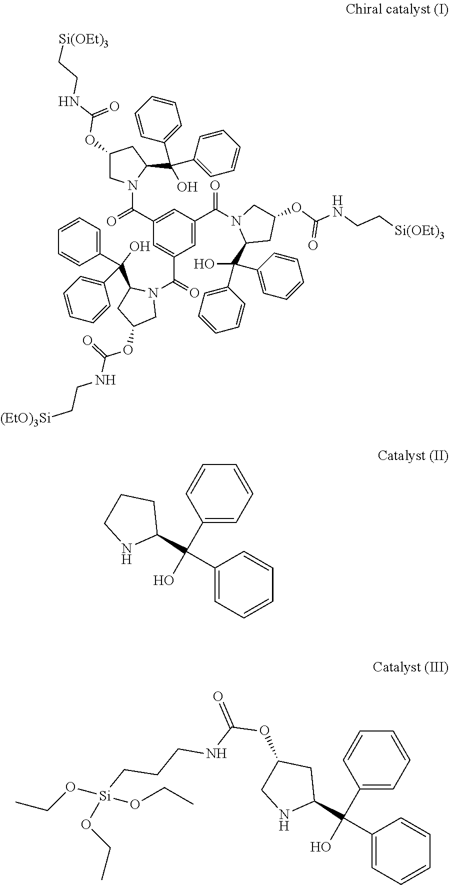

After the reaction was complete, the optical purity and conversion rate of the product were calculated. The results are shown in Table 1. The optical purity (ee) was calculated as follows:

$$Enantiomeric excess ( \% ee ) = \frac{[ R ] - [ S ]}{[ R ] + [ S ]}$$

TABLE 1

| | | | | | | R-(+) |
|---|---|---|---|---|---|---|
| (chiral) | % | | | | | conversion |
| catalyst | mol | R-(+) | S-(−) | ketone | ee | rate |
| (I) | 10% | 87.9% | 12.1% | 0% | 75.7% | 87.9% |
| (II) | 10% | 85.0% | 15.0% | 0% | 69.9% | 85.0% |
| (III) | 10% | 84.7% | 15.3% | 0% | 69.4% | 84.7% |

From the results in Table 1, it can be seen that, in the selective reduction reaction with the present chiral catalyst (I) having three silane-containing side chains added, both the optical purity and conversion rate of the product were higher than those of the product in the selective reduction reaction with the added catalysts (II) and (III).

### Example 4

Heterogeneous Chiral Catalysts for Selective Reduction Reaction

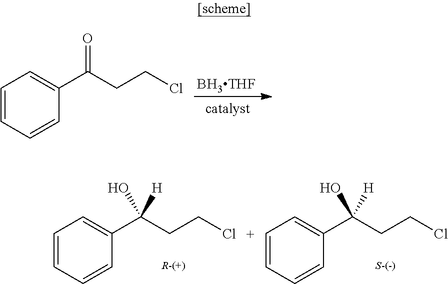

In this example, heterogeneous chiral catalysts (IV) and (V) were provided to perform the selective reduction reaction.

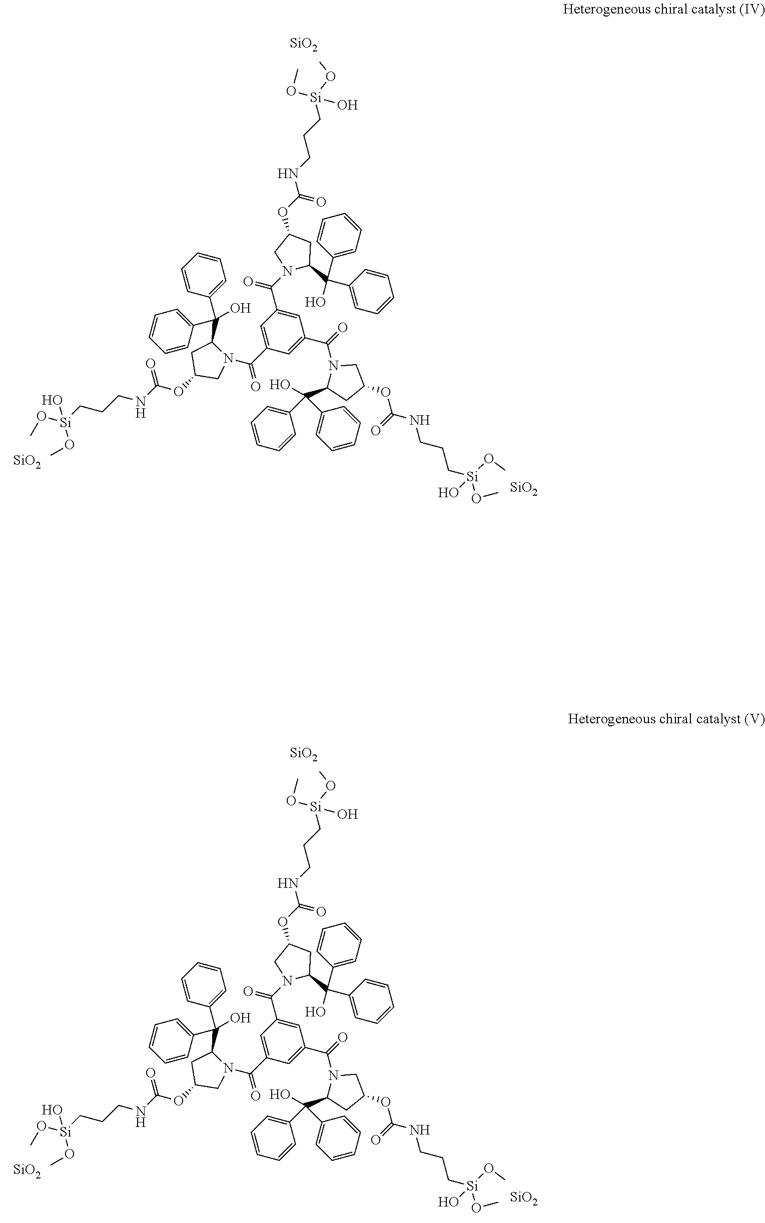

Selective Reduction Reaction:

First, 0.2M 3-chloropropiophenone solution was prepared using toluene as a solvent. Heterogeneous chiral catalyst (IV) with an amount equal to the weight of 3-chloropropiophenone was added and reacted at a temperature of 25° C. by stirring for 20 minutes. Borane tetrahydrofuran complex solution (1M) with 1.5 equivalents and a flow rate of 30 mL per hour used as a reducing agent was added. After dripping, the reaction was performed at 25° C. for 2 hours, and measured by HPLC (hexane:IPA=97: 3, 0.5 mL/min, R-(+): 11.80 min, S-(−): 13.24 min). IPLC analyzer: Multiwavelength Detector: Jasco—MD-2010 Plus, Intelligent IPLC Pum: Jasco—PU-980, Column: REGIS Whelk-O®-1-(S,S) 5 μm 100 Å, LC Column 250×4.6 mm.

Catalyst Recovery:

The solid was filtered using the FP-450 filter paper (Life Sciences) to recover heterogeneous chiral catalyst (IV). Toluene, acetone, and methanol was used to wash the recovered heterogeneous chiral catalyst (IV) in sequence. A vacuum drying step was performed at 40° C. for 8 hours, and gray solid was obtained. The selective reduction reaction and catalyst recovery method of heterogeneous chiral catalyst (V) are the same as the above steps.

After the reaction was complete, the optical purity and conversion rate of the product were calculated. The results are shown in Table 2.

TABLE 2

| heterogeneous | | | | | R-(+) |
|---|---|---|---|---|---|
| chiral catalyst | R-(+) | S-(−) | ketone | ee | conversion |
| (IV) | 83.0% | 16.1% | 0.9% | 67.5% | 83.0% |
| (V) | 88.3% | 11.4% | 0.3% | 77.2% | 88.3% |

From the results in Table 2, it can be seen that, in the selective reduction reaction with the present heterogeneous chiral catalyst connected to the substrate (for example, silicon dioxide) added, both the optical purity and conversion rate of the product can achieve good results. Even the optical purity of the product can reach 77.2%, and the conversion rate can reach 88.3%.

### Example 5

Preparation of the Chiral Catalyst (2)

Step 1

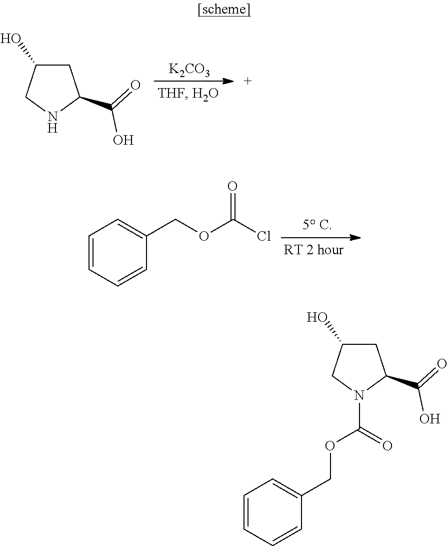

[symbol] Reagent

[symbol]

| | | molecular | | | | molar |
|---|---|---|---|---|---|---|
| item | reactant | weight | amount | | mmole | ratio |
| 1 | trans-4-hydroxy- | 131.13 | 30 | g | 228.78 | 1 |
| | L-proline | | | | | |
| 2 | potassium | 138.21 | 39.52 | g | 285.97 | 1.25 |
| | carbonate | | | | | |
| 3 | tetrahydrofuran/ | — | 120/160 | mL | — | — |
| | water | | | | | |
| 4 | benzyl | 170.59 | 35.8/120 | mL | 251.66 | 1.1 |
| | chloroformate/ | | | | | |
| | tetrahydrofuran | | | | | |

[symbol] Synthesis Steps

[symbol]

First, a 1-L double-necked reaction flask was provided, after being purged with nitrogen, items 1-3 were added to the flask and stirred until completely dissolved. Next, the reaction flask was placed in an ice bath with conduction of nitrogen to cool down, and the temperature in the ice bath was maintained at 0-5° C. Next, item 4 solvent was slowly dripped into the reaction flask. After the dripping finished, the temperature of the reaction solution was allowed to return to room temperature while stirring for 2 hours. Next, an HPLC measurement (hexane (Hex): isopropanol (IPA)=4:1 and 0.1% trifluoroacetic acid (TFA)) with flow rate of 0.5 mL/min was performed, and the retention time of the product appeared at 14.92 min. After the reaction was complete, 300 mL of H₂O was added, and extracted with 100 mL of ethanolamine (EA) each time and repeated 4 times (total 400 mL of EA). The EA layer was inspected by HPLC and no product was detected in the EA layer. Next, the aqueous layer was acidified with a 3M HCl aqueous solution to pH=2, and then extracted with 100 mL of EA each time and repeated 4 times (total 400 mL of EA). The EA layer was dehydrated with anhydrous magnesium sulfate and filtered, and then the solvent was removed at 40° C. using a rotary concentrator. Next, a vacuum drying step was performed at room temperature for 12 hours and 54 g of transparent liquid was obtained. The yield was 89.0%, and an HPLC measurement (Hex:IPA=4:1 and 0.1% TFA) with flow rate of 0.5 mL/min was performed, and the product appeared at 14.70 min [REGIS (S,S) Whelk-O1 5 μm, 4.6×150 mm]. The purity thereof was 99.2%. The target product was measured by NMR. Data are as follows: ¹H NMR (400 MHz, DMSOd₆): δ 12.63 (s, 1H, COOH), 7.36-7.28 (m, 5H), 5.10-5.00 (m, 3H, OH, PhCH₂O), 4.30-4.19 (m, 2H), 3.50-3.36 (m, 2H), 2.21-2.11 (m, 1H), 1.99-1.81 (m, 1H).

Step 2

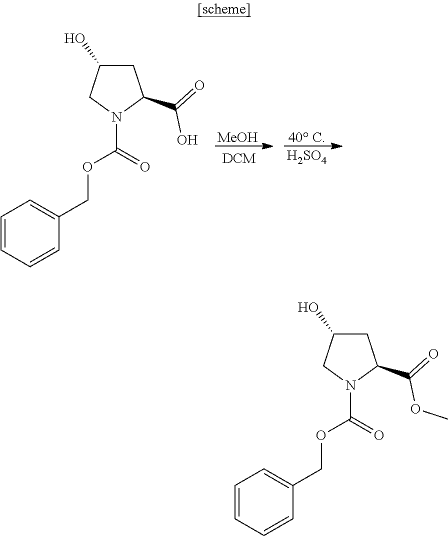 [SMILES: "MeOH" ; O=C(O)[C@@H]1C[C@@H](O)CN1C(=O)OCc1ccccc1 ; "H2SO4" ; "40° C." ; COC(=O)[C@@H]1C[C@@H](O)CN1C(=O)OCc1ccccc1]

[symbol] Reagent

[symbol]

| | | molecular | | | | molar |
|---|---|---|---|---|---|---|
| item | reactant | weight | amount | | mmole | ratio |
| 1 | product of Step 1/ | 265.26 | 54 g/75 mL | | 203.57 | 1 |
| | methanol | | | | | |
| 2 | methanol/ | — | 50/250 | mL | — | — |
| | dichloromethane | | | | | |
| 3 | sulfuric acid, | 98.08 | 1.5 | mL | 28.11 | 0.15 |
| | D = 1.84 | | | | | |

| | | | | | |
|---|---|---|---|---|---|
| 4 | potassium | 138.21 | 8.4 g/200 mL | 61.07 | 0.3 |
| | carbonate/water | | | | |

[symbol] Synthesis Steps

[symbol]

First, a 500-mL double-necked reaction flask was provided, after being purged with nitrogen, items 1-3 were added to the flask and stirred until completely dissolved. Next, the reaction flask was placed in an oil bath with conduction of nitrogen to cool down, and the temperature in the oil bath was maintained at 40-50° C. Next, the reaction was tracked by HPLC (Hex:IPA=4:1 and 0.1% TFA) with flow rate of 1.0 mL/min, and the product appeared at 11.53 min. The reaction was complete after 12 hours. After the reaction was complete, the solution was concentrated to 20-30 mL. Item 4 aqueous solution was added and extracted with 100 mL of EA each time and repeated 3 times (total 300 mL of EA). The EA layer was dehydrated with anhydrous magnesium sulfate and filtered, and then the solvent was removed at 50° C. using a rotary concentrator. Next, a vacuum drying step was performed at room temperature for 12 hours and 57.8 g of light-yellow transparent liquid was obtained. The yield was 79.5%, and an HPLC measurement (Hex:IPA=4:1 and 0.1% TFA) with flow rate of 1.0 mL/min was performed, and the product appeared at 11.507 min [REGIS (S,S) Whelk-O1 5 μm, 4.6×150 mm]. The purity thereof was 98.0%. The target product was measured by NMR. Data are as follows: H NMR (400 MHz, CDCl₃): δ 7.32-7.24 (m, 5H), 5.18-4.96 (m, 2H, PhCH2O), 4.51-4.44 (m, 2H), 3.72-3.59 (m, 3H), 3.54-3.52 (m, 2H), 2.32-2.23 (m, 1H), 2.07-1.80 (m, 1H).

Step 3

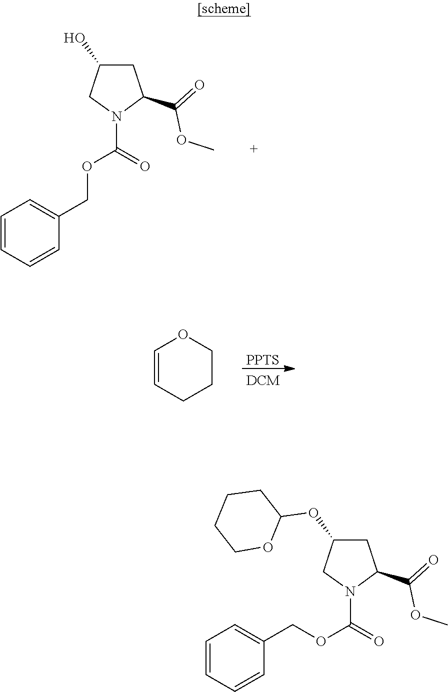

[symbol] Reagent

[symbol]

| | | molecular | | | | molar |
|---|---|---|---|---|---|---|
| item | reactant | weight | amount | | mmole | ratio |
| 1 | product of Step 2 | 279.29 | 57.9 | g | 207.3 | 1 |
| 2 | 3,4-dihydro-2H- | 84.12 | 26.15 | g | 310.97 | 1.5 |
| | pyran | | | | | |
| 3 | pyridinium p- | 251.3 | 0.52 | | 2.078 | 0.01 |
| | toluenesulfonate | | | | | |
| 4 | dichloromethane | — | 600 | mL | — | — |
| | (DCM) | | | | | |

[symbol] Synthesis Steps

[symbol]

First, a 1-L double-necked reaction flask was provided, after being purged with nitrogen, items 1-4 were added to the flask and stirred until completely dissolved. Next, the reaction flask was placed in an oil bath with conduction of nitrogen to cool down, and the temperature in the oil bath was maintained at 40° C. Next, the reaction was tracked by HPLC (Hex:IPA=4:1 and 0.1% TFA) with flow rate of 1.0 mL/min, and the product appeared at 18.613 min. The reaction was complete after 24 hours. After the reaction was complete, 300 mL of H₂O was added and extracted with DCM. The DCM layer was dehydrated with anhydrous magnesium sulfate and filtered, and then the solvent was removed at 40° C. using a rotary concentrator. Next, a vacuum drying step was performed at room temperature for 12 hours and 74.7 g of transparent liquid was obtained. The yield was 99.2%, and an HPLC measurement (Hex:IPA=4:1 and 0.1% TFA) with flow rate of 1.0 mL/min was performed, and the product appeared at 17.52 min [REGIS (S,S) Whelk-O1 5 μm, 4.6×150 mm]. The purity thereof was 99.2%. The target product was measured by NMR. Data are as follows: H NMR (400 MHz, CDCl₃): δ 7.32-7.26 (m, 5H), 5.19-4.98 (m, 2H, PhCH2O), 4.63-4.58 (m, 1H), 4.50-4.37 (m, 2H), 3.83-3.63 (m, 3H+2H), 3.54-3.41 (m, 2H), 2.45-2.29 (m, 1H), 2.14-2.01 (m, 1H), 1.75-1.62 (m, 2H), 1.58-1.39 (m, 4H).

Step 4

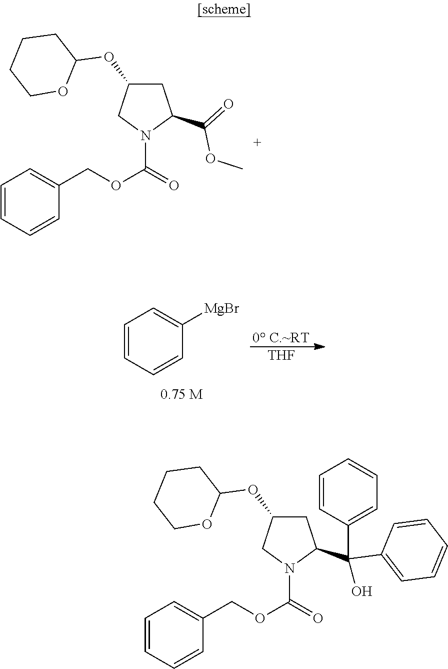

[symbol] Reagent

[symbol]

| | | molecular | | | molar |
|---|---|---|---|---|---|
| item | reactant | weight | amount | mmole | ratio |
| 1 | product of Step 3/ | 363.40 | 20 g/200 mL | 55.03 | 1 |
| | tetrahydrofuran | | | | |
| 2 | bromobenzene/ | 157.01 | 34.56 g/70 mL | 220.14 | 4 |
| | tetrahydrofuran | | | | |
| 3 | magnesium, | 24.31 | 5.62 g/10 mL | 231.12 | 4.2 |
| | turnings/ | | | | |
| | tetrahydrofuran | | | | |
| 4 | tetrahydrofuran, | — | 100 mL | — | — |
| | dry | | | | |

[symbol] Synthesis Steps

[symbol]

First, a 250-mL double-necked reaction flask was provided, after being purged with nitrogen, item 3 was added to the flask and stirred. After about 10-15 mL of item 2 was added to the reaction flask dropwise, the reaction was started by heating with a blower and boiled. After item 2 was slowly added to the system dropwise, the reaction flask was placed in an oil bath, and the temperature of the oil bath was maintained at 50-60° C. Another 500-mL double-necked reaction flask was provided, purged with nitrogen, and then item 1 was added to the flask and stirred to cool down to 0° C. to −5° C. Next, the reaction solution obtained by the above steps was placed in a feeding funnel and slowly dripped into the reaction solution, and the internal temperature was maintained at 0-10° C. After the dripping finished, the solution was heated to 40° C. and reacted for about 2 hours. Next, the reaction was tracked by HPLC (Hex:IPA=4:1 and 0.1% TFA) with flow rate of 1.0 mL/min, and the product appeared at 11.98 min. After the reaction was complete, 3M HCl aqueous solution was added to neutralize to pH=6-8, and extracted with 100 mL of EA each time and repeated 3 times (total 300 mL of EA). The EA layer was dehydrated with anhydrous magnesium sulfate and filtered, and then the solvent was removed at 50° C. using a rotary concentrator. Next, a column (3 cm in diameter) packing 40 cm (SILICYCLE Silica gel 70-230 mesh, pH=7) was provided. After impurities were eluted with EA:Hex=1:10, varied the ratio of EA:Hex to 1:4, and the product was eluted. About 30-50 mL of solvent was removed at 40° C. using a rotary concentrator. Solid was precipitated and filtered. Next, a vacuum drying step was performed at 60° C. for 12 hours, and 16.7 g of white solid was obtained. The yield was 62.3%, and an HPLC measurement (Hex:IPA=4:1 and 0.1% TFA) with flow rate of 1.0 mL/min was performed, and the product appeared at 12.053 min [REGIS (S,S) Whelk-O1 5 μm, 4.6×150 mm]. The purity thereof was 98.5%. The target product was measured by NMR. Data are as follows: H NMR (400 MHz, CDCl3): δ 7.37-7.24 (m, 15H), 5.10-4.99 (m, 2H, PhCH2O), 4.40-4.36 (d, 1H), 3.71-3.66 (m, 2H+1H), 3.39-3.32 (m, 1H), 2.23-2.09 (m, 2H), 1.70-1.69 (m, 1H), 1.62-1.24 (m, 8H).

Step 5

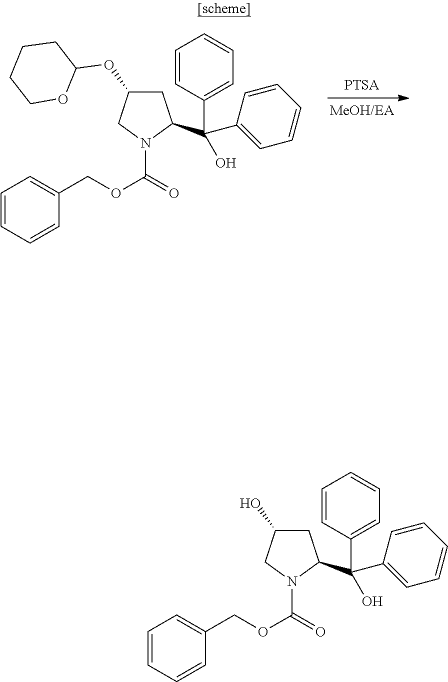 [SMILES: O=C(OCc1ccccc1)N1C[C@H](OC2CCCCO2)C[C@H]1C(O)(c1ccccc1)c1ccccc1 ; O=C(OCc1ccccc1)N1C[C@H](O)C[C@H]1C(O)(c1ccccc1)c1ccccc1]

[symbol] Reagent

[symbol]

| | | molecular | | | | molar |
|---|---|---|---|---|---|---|
| item | reactant | weight | amount | | mmole | ratio |
| 1 | product of Step 4 | 487.59 | 10 | g | 20.51 | 1 |
| 2 | p-toluenesulfonic | 190.22 | 0.04 | g | 0.205 | 0.01 |
| | acid | | | | | |
| 3 | ethyl acetate | — | 50 | mL | — | — |
| 4 | methanol | — | 50 | mL | — | — |

[symbol] Synthesis Steps

[symbol]

First, a 250-mL double-necked reaction flask was provided, after being purged with nitrogen, items 1-4 were added to the flask while stirring and heated to an internal temperature of 50-60° C. Next, the reaction was tracked by HPLC (Hex:IPA=4:1 and 0.1% TFA) with flow rate of 1.0 mL/min, and the product appeared at 8.974 min. The reaction was complete after about 4 hours. After the reaction was complete, EA was used for extraction. Each extraction was performed with 100 mL of EA and repeated 3 times (total 300 mL of EA). The EA layer was dehydrated with anhydrous magnesium sulfate and filtered, and then the solvent was removed at 50° C. using a rotary concentrator. Next, a vacuum drying step was performed at 60° C. for 12 hours and 8.3 g of white solid was obtained. The yield was 99.0%, and an HPLC measurement (Hex:IPA=4:1 and 0.1% TFA) with flow rate of 1.0 mL/min was performed, and the product appeared at 8.947 min [REGIS (S,S) Whelk-O1 5 μm, 4.6×150 mm]. The purity thereof was 88.0%. Without purification, the next step was performed. The target product was measured by NMR. Data are as follows: ¹H NMR (400 MHz, CDCl₃): δ 7.41-7.27 (m, 15H), 5.18-5.13 (m, 2H, PhCH2O), 4.97 (s, 1H), 3.93 (s, 1H), 3.62-3.59 (d, 1H), 3.08-3.06 (d, 1H), 2.20-1.98 (m, 2H), 1.62-1.60 (m, 2H).

Step 6

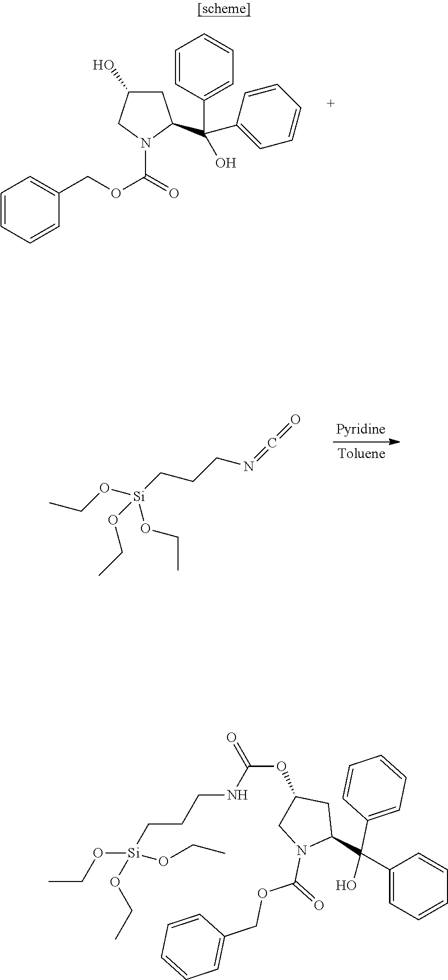 [SMILES: O=C(OCc1ccccc1)N1C[C@H](O)C[C@H]1C(O)(c1ccccc1)c1ccccc1 ; CCO[Si](CCCN=C=O)(OCC)OCC ; CCO[Si](CCCNC(=O)O[C@@H]1C[C@@H](C(O)(c2ccccc2)c2ccccc2)N(C(=O)OCc2ccccc2)C1)(OCC)OCC]

[symbol] Reagent

[symbol]

| | | molecular | | | | molar |
|---|---|---|---|---|---|---|
| item | reactant | weight | amount | | mmole | ratio |
| 1 | product of Step 5 | 403.47 | 4 | g | 9.88 | 1 |
| 2 | 3-(triethoxy- | 247.36 | 4.9 | g | 19.76 | 2 |
| | silyl)propyl | | | | | |
| | isocyanate | | | | | |
| 3 | pyridine | 79.1 | 2.34 | g | 29.64 | 3 |
| 4 | toluene | — | 40 | mL | — | — |

[symbol] Synthesis Steps

[symbol]

First, a 100-mL double-necked reaction flask was provided, after being purged with nitrogen, items 1-4 were added to the flask while stirring and heated to thermal reflux. Next, the reaction was tracked by HPLC (Hex:IPA=4:1 and 0.1% TFA) with flow rate of 1.0 mL/min, and the product appeared at 13.24 min. The reaction was complete after about 3 days. After the reaction was complete, EA was used for extraction. Each extraction was performed with 50 mL of EA and repeated 3 times (total 150 mL of EA). The EA layer was dehydrated with anhydrous magnesium sulfate and filtered, and then the solvent was removed at 50° C. using a rotary concentrator. Next, a column (3 cm in diameter) packing 40 cm (SILICYCLE Silica gel 70-230 mesh, pH=7) was provided. After impurities were eluted with EA:Hex=1:10, varied the ratio of EA:Hex to 1:4, and the product was eluted. About 30-50 mL of solvent was removed at 40° C. using a rotary concentrator. Solid was precipitated and filtered. Next, a vacuum drying step was performed at room temperature for 12 hours, and 2.75 g of transparent liquid was obtained. The yield was 45.5%, and an HPLC measurement (Hex:IPA=4:1 and 0.1% TFA) with flow rate of 1.0 mL/min was performed, and the product appeared at 13.2 min [REGIS (S,S) Whelk-O1 5 μm, 4.6×150 mm]. The purity thereof was 94.2%. The target product was measured by NMR. Data are as follows: ¹H NMR (400 MHz, CDCl3): δ 7.39-7.18 (m, 15H), 5.11-5.03 (m, 2H, PhCH2O), 4.87-4.85 (m, 2H), 4.12-4.07 (m, 1H), 3.81-3.78 (m, 6H), 3.71-3.64 (m, 1H), 3.11-3.05 (m, 2H), 2.25-2.10 (m, 2H), 1.60-1.52 (m, 2H), 1.25-1.16 (m, 9H), 0.61-0.51 (m, 2H).

Step 7

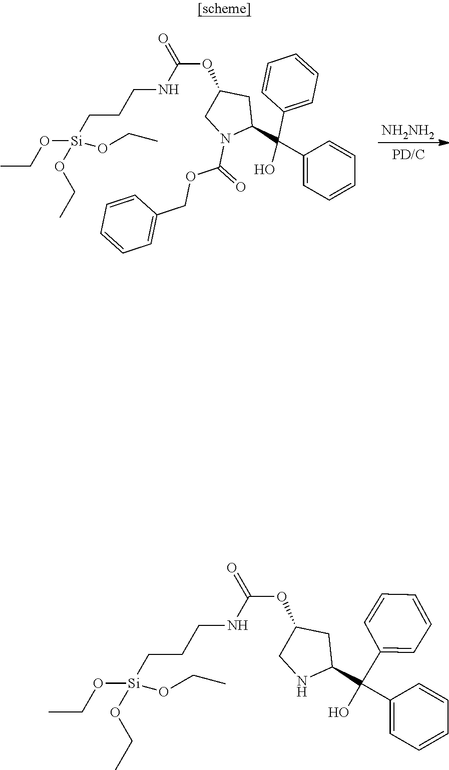

[symbol] Reagent

[symbol]

| | | molecular | | | | molar |
|---|---|---|---|---|---|---|
| item | reactant | weight | amount | | mmole | ratio |
| 1 | product of Step 6 | 650.83 | 2 | g | 3.06 | 1 |
| 2 | 5% palladium on | — | 0.2 | g | — | — |
| | carbon | | | | | |
| 3 | hydrazine monohydrate | 50.06 | 0.23 | g | 4.6 | 1.5 |
| 4 | methanol | — | 20 | mL | — | — |

[symbol] Synthesis Steps

[symbol]

First, a 100-mL double-necked reaction flask was provided, purged with nitrogen, and then items 1-4 were added to the flask while stirring and heated to an internal temperature of 50-60° C. Next, the reaction was tracked by HPLC (Hex:IPA=4:1 and 0.1% TFA) with flow rate of 1.0 mL/min, and the product appeared at 4.787 min. The reaction was complete after about 3 hours. Next, the solvent was removed at 50° C. using a rotary concentrator. Next, a column (3 cm in diameter) packing 20 cm (SILICYCLE Silica gel 70-230 mesh, pH=7) was provided using an eluent (EA:Hex=1:6). After impurities were washed out, the eluent (EA:Hex=1:1) was used to elute the product. The product was then concentrated and dried. After a vacuum drying step was performed at room temperature for 12 hours, 0.6 g of transparent liquid was obtained. The yield was 38.0%, and an HPLC measurement (Hex:IPA=4:1 and 0.1% TFA) with flow rate of 0.5 mL/min was performed, and the product appeared at 10.2 min [REGIS (S,S) Whelk-O1 5 μm, 4.6×150 mm]. The purity thereof was 93.0%. The target product was measured by NMR. Data are as follows: H NMR (400 MHz, CDCl₃): δ 7.58-7.53 (m, 2H), 7.44-7.42 (m, 2H), 7.30-7.11 (m, 6H), 5.06 (s, 1H), 4.90 (s, 1H), 4.51-4.47 (m, 1H), 3.82-3.77 (m, 6H), 3.26-3.22 (m, 2H), 3.15-3.04 (m, 2H), 1.63-1.51 (m, 4H), 1.22-1.19 (m, 9H), 0.62-0.58 (m, 2H).

Step 8

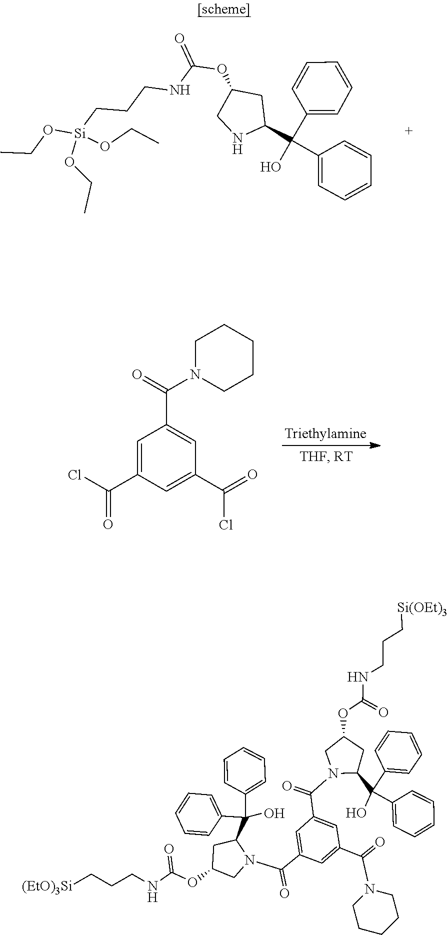

[symbol]

Reagent

[symbol]

| | | molecular | | | | molar |
|---|---|---|---|---|---|---|
| item | reactant | weight | amount | | mmole | ratio |
| 1 | product of Step 7 | 516.70 | 0.516 | g | 1.0 | 2.2 |
| 2 | 1-piperidinyl- | 314.16 | 0.143 | g | 0.455 | 1 |
| | carbonyl-3,5- | | | | | |
| | benzenedicarbonyl | | | | | |
| | dichloride | | | | | |
| 3 | tetrahydrofuran | — | 20 | mL | — | — |

| | | | | | |
|---|---|---|---|---|---|
| 4 | triethylamine, | 101.19 | 0.42 g/0.57 mL | 4.1 | 9 |
| | D = 0.726 | | | | |

[symbol] Synthesis Steps

[symbol]

First, a 100-mL double-necked reaction flask was provided, purged with nitrogen, and then items 1-3 were added to the flask with stirring. After item 4 was slowly dripped into the above reaction solution, solid salts were precipitated. The reaction was complete after about 24 hours. After the reaction was complete, EA was used for extraction. Each extraction was performed with 100 mL of EA and repeated 3 times (total 300 mL of EA). The EA layer was dehydrated with anhydrous magnesium sulfate and filtered, and then the solvent was removed at 35° C. using a rotary concentrator. The filtrate was dissolved in 10 mL of acetone and recrystallized using hexane. The product was filtered using the FP-450 filter paper (Life Sciences). Next, a vacuum drying step was performed at 40° C. for 12 hours, and 0.46 g of white solid was obtained. The yield was 80.1%, and an HPLC measurement (Hex:IPA=4:1 and 0.1% TFA) with flow rate of 0.5 mL/min was performed, and the product appeared at 6.46 min [REGIS (S,S) Whelk-O1 5 μm, 4.6×150 mm]. The purity thereof was 98.6%. The target product was measured by NMR. Data are as follows: ¹H NMR (400 MHz, CDCl₃): δ 7.58-7.51 (m, 4H), 7.44-7.38 (m, 4H), 7.38-7.02 (m, 18H+3H), 5.11 (s, 2H), 4.98 (s, 2H), 4.51-4.46 (m, 2H), 3.81-3.72 (m, 12H), 3.38-3.42 (m, 4H), 3.26-3.31 (m, 4H), 3.20-2.94 (m, 4H), 1.69-1.50 (m, 8H+6H), 1.23-1.16 (m, 18H), 0.61-0.56 (m, 4H).

### Example 6

Preparation of the Chiral Catalyst (3)

Step 1

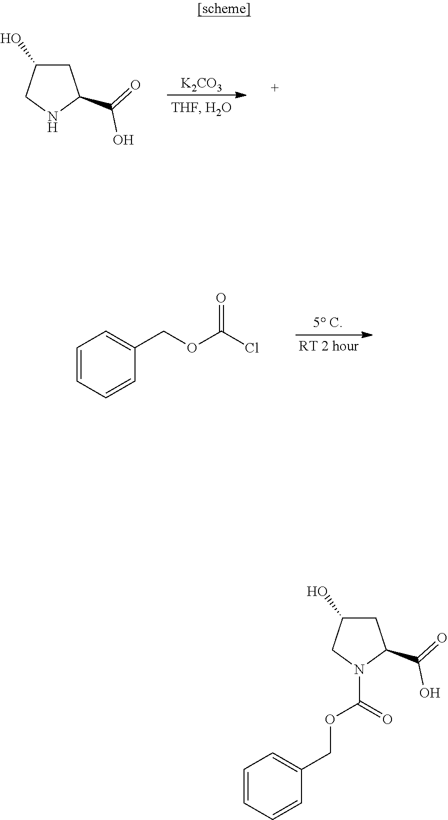

[symbol] Reagent

[symbol]

| | | molecular | | | | molar |
|---|---|---|---|---|---|---|
| item | reactant | weight | amount | | mmole | ratio |
| 1 | trans-4-hydroxy- | 131.13 | 30 | g | 228.78 | 1 |
| | L-proline | | | | | |
| 2 | potassium | 138.21 | 39.52 | g | 285.97 | 1.25 |
| | carbonate | | | | | |
| 3 | tetrahydrofuran/ | — | 120/160 | mL | — | — |
| | water | | | | | |
| 4 | benzyl | 170.59 | 35.8/120 | mL | 251.66 | 1.1 |
| | chloroformate/ | | | | | |
| | tetrahydrofuran | | | | | |

[symbol] Synthesis Steps

[symbol]

First, a 1-L double-necked reaction flask was provided, after being purged with nitrogen, items 1-3 were added to the flask and stirred until completely dissolved. Next, the reaction flask was placed in an ice bath with conduction of nitrogen to cool down, and the temperature in the ice bath was maintained at 0-5° C. Next, item 4 solvent was slowly dripped into the reaction flask. After the dripping finished, the temperature of the reaction solution was allowed to return to room temperature while stirring for 2 hours. Next, an HPLC measurement (hexane (Hex): isopropanol (IPA)=4:1 and 0.1% trifluoroacetic acid (TFA)) with flow rate of 0.5 mL/min was performed, and the retention time of the product appeared at 14.92 min. After the reaction was complete, 300 mL of H₂O was added, and extracted with 100 mL of ethanolamine (EA) each time and repeated 4 times (total 400 mL of EA). The EA layer was inspected by HPLC and no product was detected in the EA layer. Next, the aqueous layer was acidified with a 3M HCl aqueous solution to pH=2, and then extracted with 100 mL of EA each time and repeated 4 times (total 400 mL of EA). The EA layer was dehydrated with anhydrous magnesium sulfate and filtered, and then the solvent was removed at 40° C. using a rotary concentrator. Next, a vacuum drying step was performed at room temperature for 12 hours and 54 g of transparent liquid was obtained. The yield was 89.0%, and an HPLC measurement (Hex:IPA=4:1 and 0.1% TFA) with flow rate of 0.5 mL/min was performed, and the product appeared at 14.70 min [REGIS (S,S) Whelk-O1 5 μm, 4.6×150 mm]. The purity thereof was 99.2%. The target product was measured by NMR. Data are as follows: ¹H NMR (400 MHz, DMSOd₆): δ 12.63 (s, 1H, COOH), 7.36-7.28 (m, 5H), 5.10-5.00 (m, 3H, OH, PhCH₂O), 4.30-4.19 (m, 2H), 3.50-3.36 (m, 2H), 2.21-2.11 (m, 1H), 1.99-1.81 (m, 1H).

Step 2

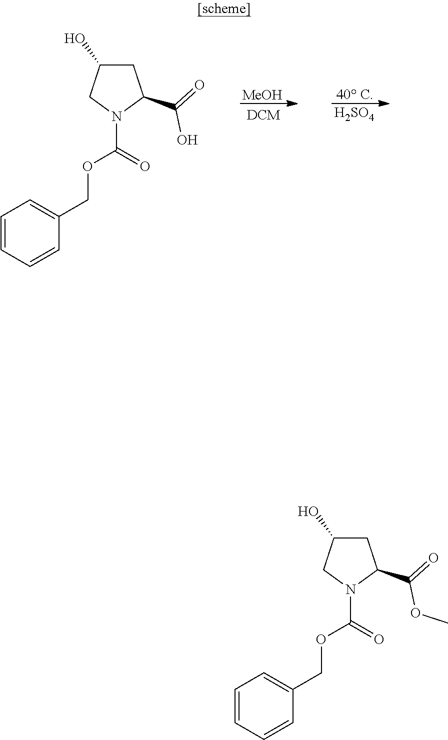 [SMILES: "MeOH" ; O=C(O)[C@@H]1C[C@@H](O)CN1C(=O)OCc1ccccc1 ; "H2SO4" ; "40° C." ; COC(=O)[C@@H]1C[C@@H](O)CN1C(=O)OCc1ccccc1]

[symbol] Reagent

[symbol]

| | | molecular | | | | molar |
|---|---|---|---|---|---|---|
| item | reactant | weight | amount | | mmole | ratio |
| 1 | product of Step 1/ | 265.26 | 54 g/75 mL | | 203.57 | 1 |
| | methanol | | | | | |
| 2 | methanol/ | — | 50/250 | mL | — | — |
| | dichloromethane | | | | | |
| 3 | sulfuric acid, | 98.08 | 1.5 | mL | 28.11 | 0.15 |
| | D = 1.84 | | | | | |

| | | | | | |
|---|---|---|---|---|---|
| 4 | potassium | 138.21 | 8.4 g/200 mL | 61.07 | 0.3 |
| | carbonate/water | | | | |

[symbol] Synthesis Steps

[symbol]

First, a 500-mL double-necked reaction flask was provided, after being purged with nitrogen, items 1-3 were added to the flask and stirred until completely dissolved. Next, the reaction flask was placed in an oil bath with conduction of nitrogen to cool down, and the temperature in the oil bath was maintained at 40-50° C. Next, the reaction was tracked by HPLC (Hex:IPA=4:1 and 0.1% TFA) with flow rate of 1.0 mL/min, and the product appeared at 11.53 min. The reaction was complete after 12 hours. After the reaction was complete, the solution was concentrated to 20-30 mL. Item 4 aqueous solution was added and extracted with 100 mL of EA each time and repeated 3 times (total 300 mL of EA). The EA layer was dehydrated with anhydrous magnesium sulfate and filtered, and then the solvent was removed at 50° C. using a rotary concentrator. Next, a vacuum drying step was performed at room temperature for 12 hours and 57.8 g of light-yellow transparent liquid was obtained. The yield was 79.5%, and an HPLC measurement (Hex:IPA=4:1 and 0.1% TFA) with flow rate of 1.0 mL/min was performed, and the product appeared at 11.507 min [REGIS (S,S) Whelk-O1 5 μm, 4.6×150 mm]. The purity thereof was 98.0%. The target product was measured by NMR. Data are as follows: H NMR (400 MHz, CDCl₃): δ 7.32-7.24 (m, 5H), 5.18-4.96 (m, 2H, PhCH2O), 4.51-4.44 (m, 2H), 3.72-3.59 (m, 3H), 3.54-3.52 (m, 2H), 2.32-2.23 (m, 1H), 2.07-1.80 (m, 1H).

Step 3

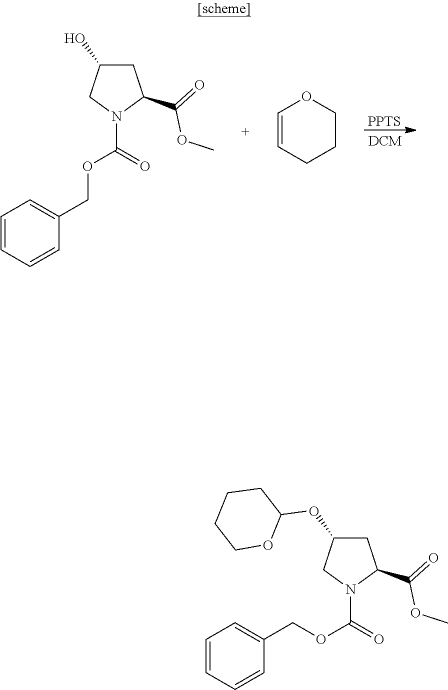

[symbol] Reagent

[symbol]

| | | molecular | | | | molar |
|---|---|---|---|---|---|---|
| item | reactant | weight | amount | | mmole | ratio |
| 1 | product of Step 2 | 279.29 | 57.9 | g | 207.3 | 1 |
| 2 | 3,4-dihydro-2H- | 84.12 | 26.15 | g | 310.97 | 1.5 |
| | pyran | | | | | |
| 3 | pyridinium p- | 251.3 | 0.52 | | 2.078 | 0.01 |
| | toluenesulfonate | | | | | |
| 4 | di chloromethane | — | 600 | mL | — | — |

[symbol] Synthesis Steps

[symbol]

First, a 1-L double-necked reaction flask was provided, after being purged with nitrogen, items 1-4 were added to the flask and stirred until completely dissolved. Next, the reaction flask was placed in an oil bath with conduction of nitrogen to cool down, and the temperature in the oil bath was maintained at 40° C. Next, the reaction was tracked by IPLC (Hex:IPA=4:1 and 0.1% TFA) with flow rate of 1.0 mL/min, and the product appeared at 18.613 min. The reaction was complete after 24 hours. After the reaction was complete, 300 mL of H₂O was added and extracted with DCM. The DCM layer was dehydrated with anhydrous magnesium sulfate and filtered, and then the solvent was removed at 40° C. using a rotary concentrator. Next, a vacuum drying step was performed at room temperature for 12 hours and 74.7 g of transparent liquid was obtained. The yield was 99.2%, and an HPLC measurement (Hex:IPA=4:1 and 0.1% TFA) with flow rate of 1.0 mL/min was performed, and the product appeared at 17.52 min [REGIS (S,S) Whelk-O1 5 μm, 4.6×150 mm]. The purity thereof was 99.2%. The target product was measured by NMR. Data are as follows: ¹H NMR (400 MHz, CDCl₃): δ 7.32-7.26 (m, 5H), 5.19-4.98 (m, 2H, PhCH2O), 4.63-4.58 (m, 1H), 4.50-4.37 (m, 2H), 3.83-3.63 (m, 3H+2H), 3.54-3.41 (m, 2H), 2.45-2.29 (m, 1H), 2.14-2.01 (m, 1H), 1.75-1.62 (m, 2H), 1.58-1.39 (m, 4H).

Step 4

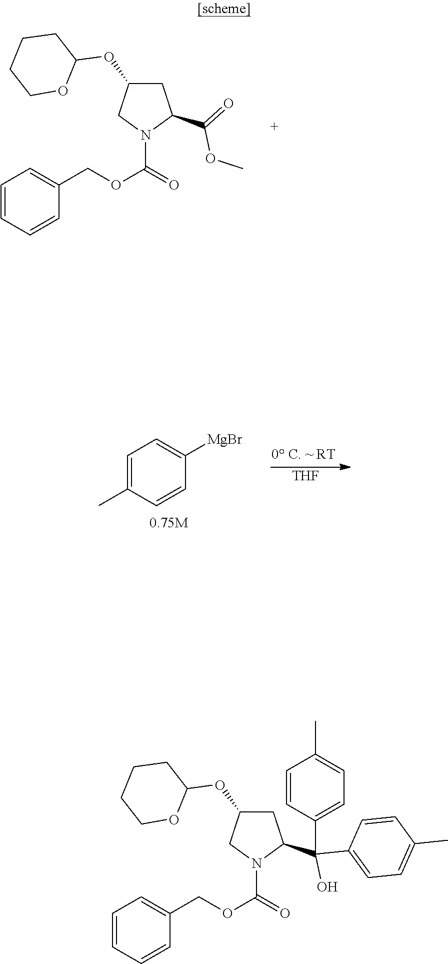

[symbol] Reagent

[symbol]

| | | molecular | | | molar |
|---|---|---|---|---|---|
| item | reactant | weight | amount | mmole | ratio |
| 1 | product of Step | 363.40 | 20 g/200 mL | 55.03 | 1 |
| | 3/tetrahydrofuran | | | | |
| 2 | bromobenzene/ | 171.04 | 37.65 g/70 mL | 220.14 | 4 |
| | tetrahydrofuran | | | | |
| 3 | magnesium, | 24.31 | 5.62 g/10 mL | 231.12 | 4.2 |
| | turnings/ | | | | |
| | tetrahydrofuran | | | | |
| 4 | tetrahydrofuran, | — | 100 mL | — | — |
| | dry | | | | |

[symbol] Synthesis Steps

[symbol]

The synthesis steps are the same as Step 4 of Example 1.

Step 5

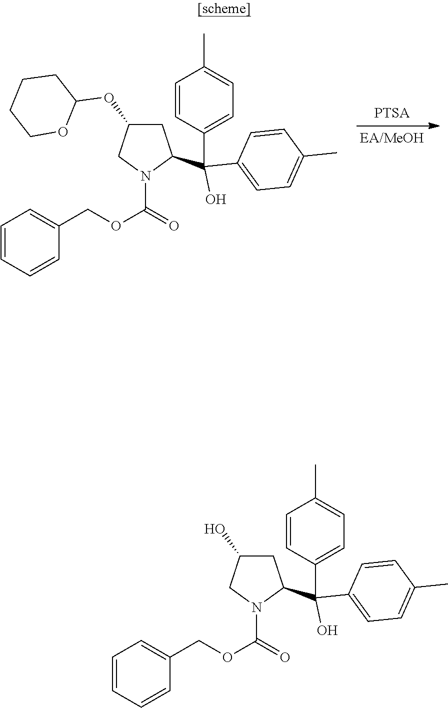 [SMILES: Cc1ccc(C(O)(c2ccc(C)cc2)[C@@H]2C[C@@H](OC3CCCCO3)CN2C(=O)OCc2ccccc2)cc1 ; Cc1ccc(C(O)(c2ccc(C)cc2)[C@@H]2C[C@@H](O)CN2C(=O)OCc2ccccc2)cc1]

[symbol] Reagent

[symbol]

| | | molecular | | | | molar |
|---|---|---|---|---|---|---|
| item | reactant | weight | amount | | mmole | ratio |
| 1 | product of Step 4 | 515.64 | 10.58 | g | 20.51 | 1 |
| 2 | p-toluenesulfonic | 190.22 | 0.04 | g | 0.205 | 0.01 |
| | acid | | | | | |
| 3 | ethyl acetate | — | 50 | mL | — | — |
| 4 | methanol | — | 50 | mL | — | — |

[symbol] Synthesis Steps

[symbol]

The synthesis steps are the same as Step 5 of Example 1.

Step 6

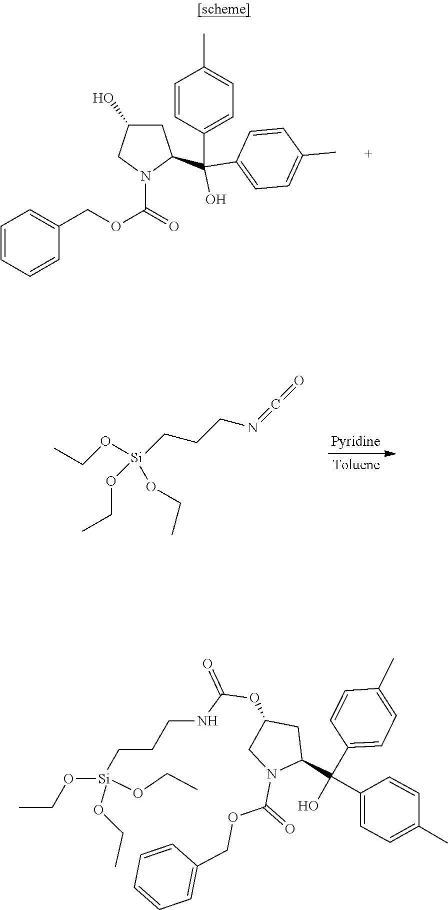

[symbol]

Reagent

[symbol]

| | | molecular | | | | molar |
|---|---|---|---|---|---|---|
| item | reactant | weight | amount | | mmole | ratio |
| 1 | product of Step 5 | 431.52 | 4.26 | g | 9.88 | 1 |
| 2 | 3-(triethoxy- | 247.36 | 4.9 | g | 19.76 | 2 |
| | silyl)propyl | | | | | |
| | isocyanate | | | | | |
| 3 | pyridine | 79.1 | 2.34 | g | 29.64 | 3 |
| 4 | toluene | — | 40 | mL | — | — |

[symbol] Synthesis Steps

[symbol]

The synthesis steps are the same as Step 6 of Example 1.

Step 7

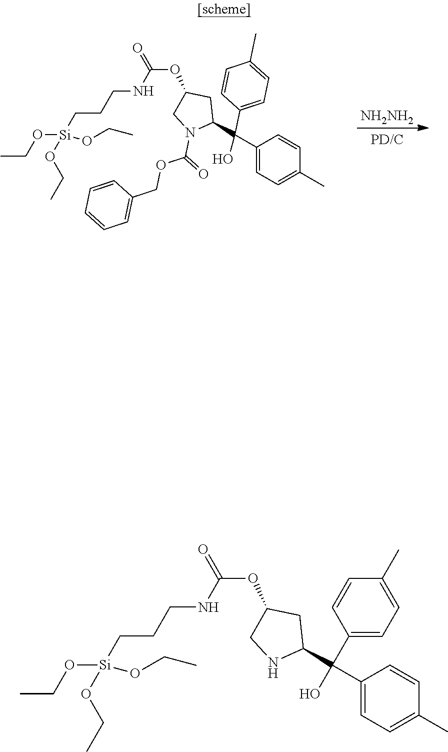

[symbol] Reagent

[symbol]

| | | molecular | | | | molar |
|---|---|---|---|---|---|---|
| item | reactant | weight | amount | | mmole | ratio |
| 1 | product of Step 6 | 678.89 | 2.08 | g | 3.06 | 1 |
| 2 | 5% palladium on | — | 0.2 | g | — | — |
| | carbon | | | | | |
| 3 | hydrazine | 50.06 | 0.23 | g | 4.6 | 1.5 |
| | monohydrate | | | | | |
| 4 | methanol | — | 20 | mL | — | — |

[symbol] Synthesis Steps

[symbol]

The synthesis steps are the same as Step 7 of Example 1.

Step 8

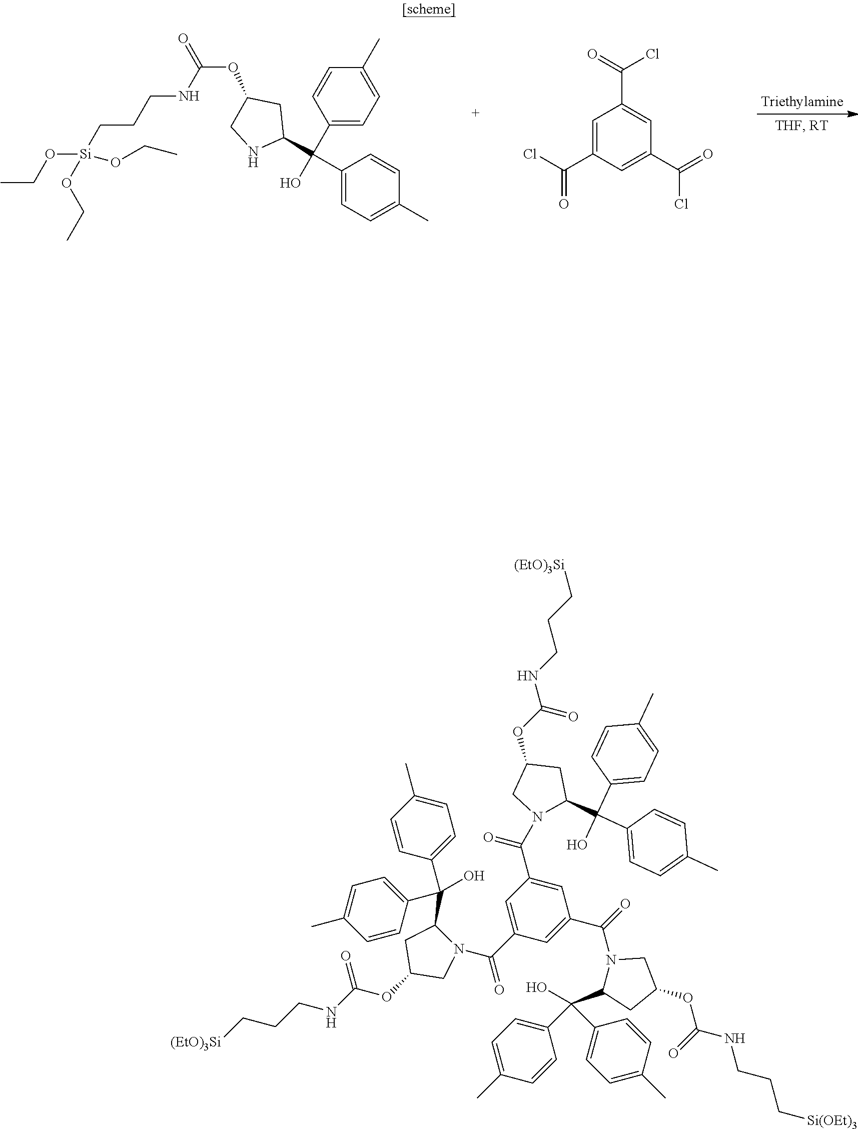

[symbol]

Reagent

[symbol]

| | | molecular | | | | molar |
|---|---|---|---|---|---|---|
| item | reactant | weight | amount | | mmole | ratio |
| 1 | product of Step 7 | 544.75 | 0.56 | g | 1.025 | 3.3 |
| 2 | 1,3,5-benzene- | 265.47 | 0.082 | g | 0.310 | 1 |
| | tricarbonyl | | | | | |
| | trichloride, | | | | | |
| | D = 1.487 | | | | | |
| 3 | tetrahydrofuran | — | 20 | mL | — | — |

| | | | | | |
|---|---|---|---|---|---|
| 4 | triethylamine, | 101.19 | 0.93 g/1.3 mL | 9.225 | 9 |
| | D = 0.726 | | | | |

[symbol] Synthesis Steps

[symbol]

The synthesis steps are the same as Step 8 of Example 1.

### Example 7

Preparation of the Chiral Catalyst (4)

Step 1

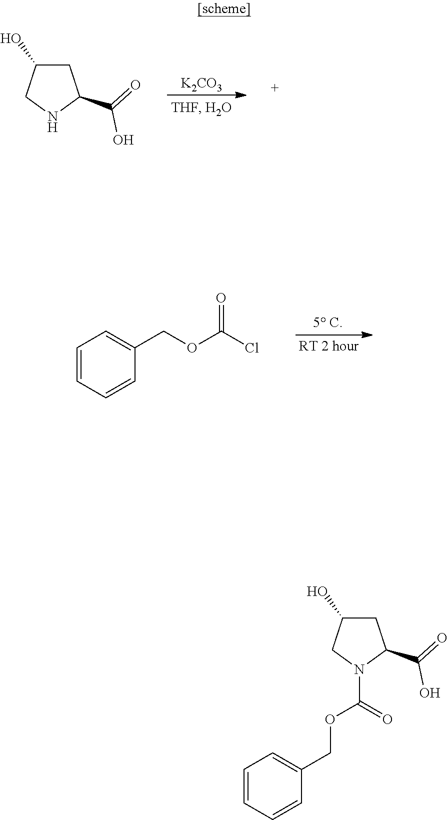

[symbol] Reagent

[symbol]

| | | molecular | | | | molar |
|---|---|---|---|---|---|---|
| item | reactant | weight | amount | | mmole | ratio |
| 1 | trans-4-hydroxy- | 131.13 | 30 | g | 228.78 | 1 |
| | L-proline | | | | | |
| 2 | potassium | 138.21 | 39.52 | g | 285.97 | 1.25 |
| | carbonate | | | | | |
| 3 | tetrahydrofuran/ | — | 120/160 | mL | — | — |
| | water | | | | | |
| 4 | benzyl | 170.59 | 35.8/120 | mL | 251.66 | 1.1 |
| | chloroformate/ | | | | | |
| | tetrahydrofuran | | | | | |

[symbol] Synthesis Steps

[symbol]

First, a 1-L double-necked reaction flask was provided, after being purged with nitrogen, items 1-3 were added to the flask and stirred until completely dissolved. Next, the reaction flask was placed in an ice bath with conduction of nitrogen to cool down, and the temperature in the ice bath was maintained at 0-5° C. Next, item 4 solvent was slowly dripped into the reaction flask. After the dripping finished, the temperature of the reaction solution was allowed to return to room temperature while stirring for 2 hours. Next, an HPLC measurement (hexane (Hex): isopropanol (IPA)=4:1 and 0.1% trifluoroacetic acid (TFA)) with flow rate of 0.5 mL/min was performed, and the retention time of the product appeared at 14.92 min. After the reaction was complete, 300 mL of H₂O was added, and extracted with 100 mL of ethanolamine (EA) each time and repeated 4 times (total 400 mL of EA). The EA layer was inspected by HPLC and no product was detected in the EA layer. Next, the aqueous layer was acidified with a 3M HCl aqueous solution to pH=2, and then extracted with 100 mL of EA each time and repeated 4 times (total 400 mL of EA). The EA layer was dehydrated with anhydrous magnesium sulfate and filtered, and then the solvent was removed at 40° C. using a rotary concentrator. Next, a vacuum drying step was performed at room temperature for 12 hours and 54 g of transparent liquid was obtained. The yield was 89.0%, and an HPLC measurement (Hex:IPA=4:1 and 0.1% TFA) with flow rate of 0.5 mL/min was performed, and the product appeared at 14.70 min [REGIS (S,S) Whelk-O1 5 μm, 4.6×150 mm]. The purity thereof was 99.2%. The target product was measured by NMR. Data are as follows: ¹H NMR (400 MHz, DMSOd₆): δ 12.63 (s, 1H, COOH), 7.36-7.28 (m, 5H), 5.10-5.00 (m, 3H, OH, PhCH₂O), 4.30-4.19 (m, 2H), 3.50-3.36 (m, 2H), 2.21-2.11 (m, 1H), 1.99-1.81 (m, 1H).

Step 2

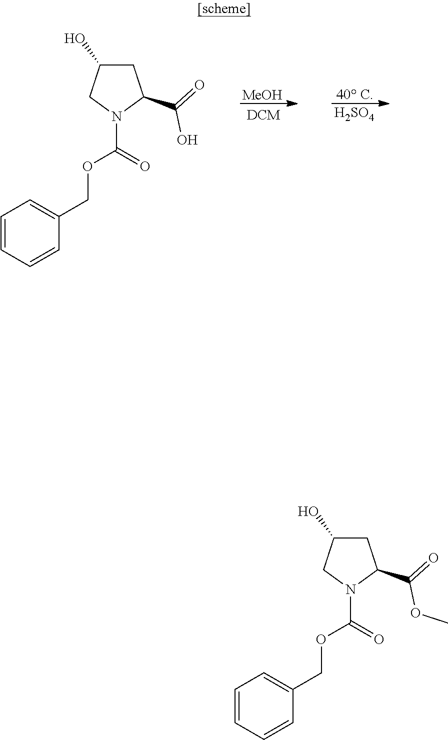 [SMILES: "MeOH" ; O=C(O)[C@@H]1C[C@@H](O)CN1C(=O)OCc1ccccc1 ; "H2SO4" ; "40° C." ; COC(=O)[C@@H]1C[C@@H](O)CN1C(=O)OCc1ccccc1]

[symbol] Reagent

[symbol]

| | | molecular | | | | molar |
|---|---|---|---|---|---|---|
| item | reactant | weight | amount | | mmole | ratio |
| 1 | product of Step 1/ | 265.26 | 54 g/75 mL | | 203.57 | 1 |
| | methanol | | | | | |
| 2 | methanol/ | — | 50/250 | mL | — | — |
| | dichloromethane | | | | | |
| 3 | sulfuric acid, | 98.08 | 1.5 | mL | 28.11 | 0.15 |
| | D = 1.84 | | | | | |

| | | | | | |
|---|---|---|---|---|---|
| 4 | potassium | 138.21 | 8.4 g/200 mL | 61.07 | 0.3 |
| | carbonate/water | | | | |

[symbol] Synthesis Steps

[symbol]

First, a 500-mL double-necked reaction flask was provided, after being purged with nitrogen, items 1-3 were added to the flask and stirred until completely dissolved. Next, the reaction flask was placed in an oil bath with conduction of nitrogen to cool down, and the temperature in the oil bath was maintained at 40-50° C. Next, the reaction was tracked by HPLC (Hex:IPA=4:1 and 0.1% TFA) with flow rate of 1.0 mL/min, and the product appeared at 11.53 min. The reaction was complete after 12 hours. After the reaction was complete, the solution was concentrated to 20-30 mL. Item 4 aqueous solution was added and extracted with 100 mL of EA each time and repeated 3 times (total 300 mL of EA). The EA layer was dehydrated with anhydrous magnesium sulfate and filtered, and then the solvent was removed at 50° C. using a rotary concentrator. Next, a vacuum drying step was performed at room temperature for 12 hours and 57.8 g of light-yellow transparent liquid was obtained. The yield was 79.5%, and an HPLC measurement (Hex:IPA=4:1 and 0.1% TFA) with flow rate of 1.0 mL/min was performed, and the product appeared at 11.507 min [REGIS (S,S) Whelk-O1 5 μm, 4.6×150 mm]. The purity thereof was 98.0%. The target product was measured by NMR. Data are as follows: H NMR (400 MHz, CDCl₃): δ 7.32-7.24 (m, 5H), 5.18-4.96 (m, 2H, PhCH2O), 4.51-4.44 (m, 2H), 3.72-3.59 (m, 3H), 3.54-3.52 (m, 2H), 2.32-2.23 (m, 1H), 2.07-1.80 (m, 1H).

Step 3

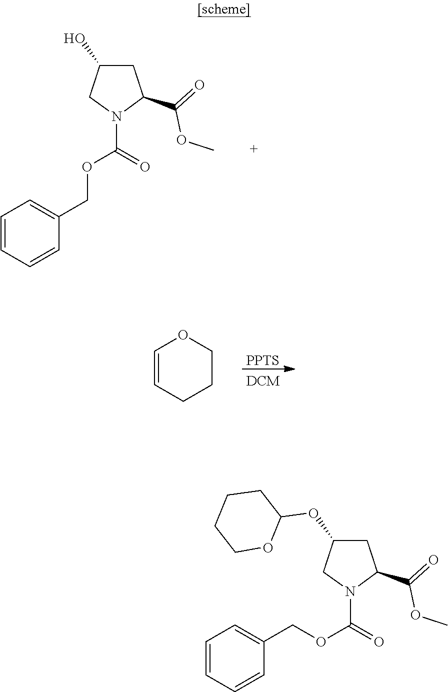

[symbol] Reagent

[symbol]

| | | molecular | | | | molar |
|---|---|---|---|---|---|---|
| item | reactant | weight | amount | | mmole | ratio |
| 1 | product of Step 2 | 279.29 | 57.9 | g | 207.3 | 1 |
| 2 | 3,4-dihydro-2H- | 84.12 | 26.15 | g | 310.97 | 1.5 |
| | pyran | | | | | |
| 3 | pyridinium p-tol | 251.3 | 0.52 | | 2.078 | 0.01 |
| | uenesulfonate | | | | | |
| 4 | dichloromethane | — | 600 | mL | — | — |

[symbol] Synthesis Steps

[symbol]

First, a 1-L double-necked reaction flask was provided, after being purged with nitrogen, items 1-4 were added to the flask and stirred until completely dissolved. Next, the reaction flask was placed in an oil bath with conduction of nitrogen to cool down, and the temperature in the oil bath was maintained at 40° C. Next, the reaction was tracked by HPLC (Hex:IPA=4:1 and 0.1% TFA) with flow rate of 1.0 mL/min, and the product appeared at 18.613 min. The reaction was complete after 24 hours. After the reaction was complete, 300 mL of H₂O was added and extracted with DCM. The DCM layer was dehydrated with anhydrous magnesium sulfate and filtered, and then the solvent was removed at 40° C. using a rotary concentrator. Next, a vacuum drying step was performed at room temperature for 12 hours and 74.7 g of transparent liquid was obtained. The yield was 99.2%, and an HPLC measurement (Hex:IPA=4:1 and 0.1% TFA) with flow rate of 1.0 mL/min was performed, and the product appeared at 17.52 min [REGIS (S,S) Whelk-O1 5 μm, 4.6×150 mm]. The purity thereof was 99.2%. The target product was measured by NMR. Data are as follows: H NMR (400 MHz, CDCl₃): δ 7.32-7.26 (m, 5H), 5.19-4.98 (m, 2H, PhCH2O), 4.63-4.58 (m, 1H), 4.50-4.37 (m, 2H), 3.83-3.63 (m, 3H+2H), 3.54-3.41 (m, 2H), 2.45-2.29 (m, 1H), 2.14-2.01 (m, 1H), 1.75-1.62 (m, 2H), 1.58-1.39 (m, 4H).

Step 4

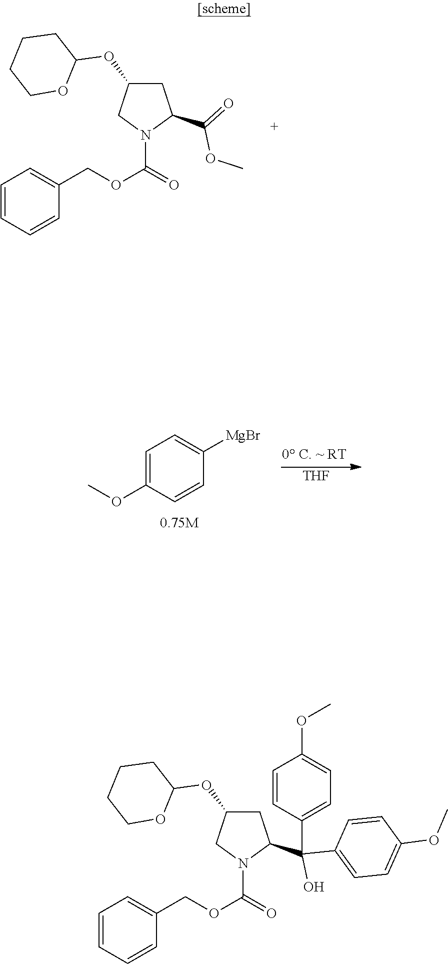

[symbol] Reagent

[symbol]

| | | molecular | | | molar |
|---|---|---|---|---|---|
| item | reactant | weight | amount | mmole | ratio |
| 1 | product of Step 3/ | 363.40 | 20 g/200 mL | 55.03 | 1 |
| | tetrahydrofuran | | | | |
| 2 | bromobenzene/ | 171.04 | 37.65 g/70 mL | 220.14 | 4 |
| | tetrahydrofuran | | | | |
| 3 | magnesium, | 24.31 | 5.62 g/10 mL | 231.12 | 4.2 |
| | turnings/ | | | | |
| | tetrahydrofuran | | | | |
| 4 | tetrahydrofuran, | — | 100 mL | — | — |
| | dry | | | | |

[symbol] Synthesis Steps

[symbol]

The synthesis steps are the same as Step 4 of Example 1.

Step 5

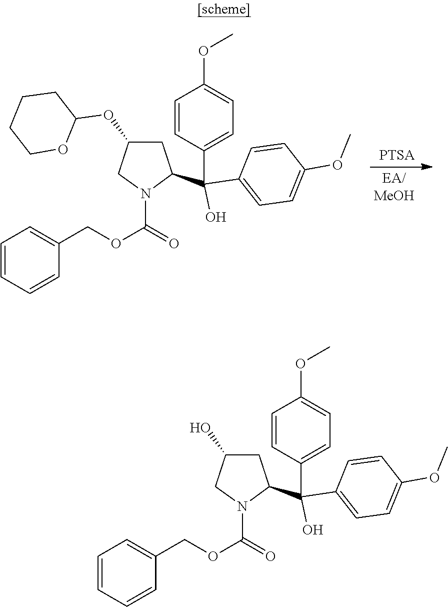 [SMILES: COc1ccc(C(O)(c2ccc(OC)cc2)[C@@H]2C[C@@H](OC3CCCCO3)CN2C(=O)OCc2ccccc2)cc1 ; COc1ccc(C(O)(c2ccc(OC)cc2)[C@@H]2C[C@@H](O)CN2C(=O)OCc2ccccc2)cc1]

[symbol] Reagent

[symbol]

| | | molecular | | | | molar |
|---|---|---|---|---|---|---|
| item | reactant | weight | amount | | mmole | ratio |
| 1 | product of Step 4 | 547.64 | 11.23 | g | 20.51 | 1 |
| 2 | p-toluenesulfonic | 190.22 | 0.04 | g | 0.205 | 0.01 |
| | acid | | | | | |
| 3 | ethyl acetate | — | 50 | mL | — | — |
| 4 | methanol | — | 50 | mL | — | — |

[symbol] Synthesis Steps

[symbol]

The synthesis steps are the same as Step 5 of Example 1.

Step 6

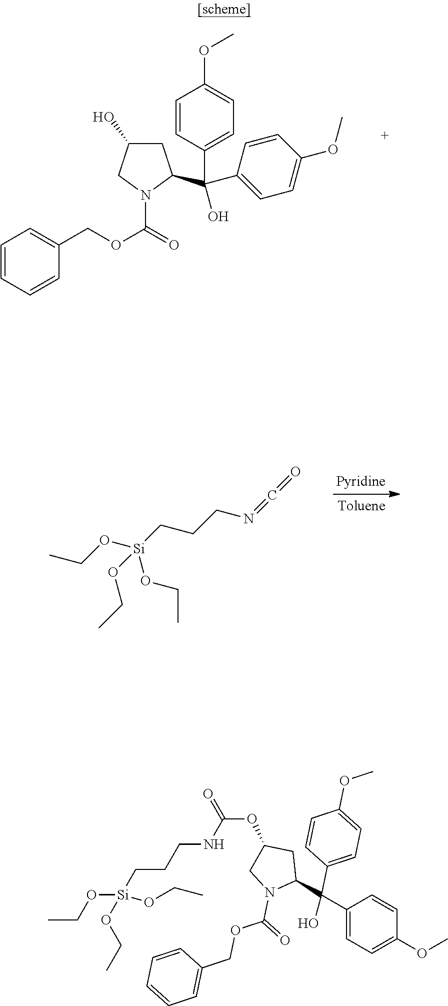

[symbol]

Reagent

[symbol]

| | | molecular | | | | molar |
|---|---|---|---|---|---|---|
| item | reactant | weight | amount | | mmole | ratio |
| 1 | product of Step 5 | 463.52 | 4.58 | g | 9.88 | 1 |
| 2 | 3-(triethoxy- | 247.36 | 4.9 | g | 19.76 | 2 |
| | silyl)propyl | | | | | |
| | isocyanate | | | | | |
| 3 | pyridine | 79.1 | 2.34 | g | 29.64 | 3 |
| 4 | toluene | — | 40 | mL | — | — |

[symbol] Synthesis Steps

[symbol]

The synthesis steps are the same as Step 6 of Example 1.

Step 7

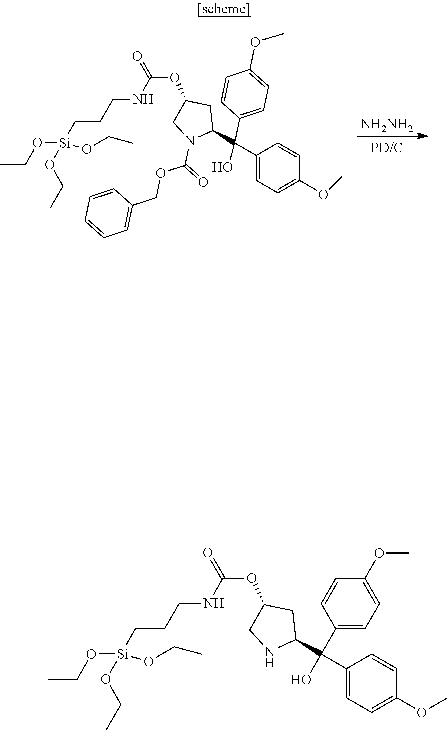

[symbol] Reagent

[symbol]

| | | molecular | | | | molar |
|---|---|---|---|---|---|---|
| item | reactant | weight | amount | | mmole | ratio |
| 1 | product of Step 6 | 710.89 | 2.18 | g | 3.06 | 1 |
| 2 | 5% palladium on | — | 0.2 | g | — | — |
| | carbon | | | | | |
| 3 | hydrazine | 50.06 | 0.23 | g | 4.6 | 1.5 |
| | monohydrate | | | | | |
| 4 | methanol | — | 20 | mL | — | — |

[symbol] Synthesis Steps

[symbol]

The synthesis steps are the same as Step 7 of Example 1.

Step 8

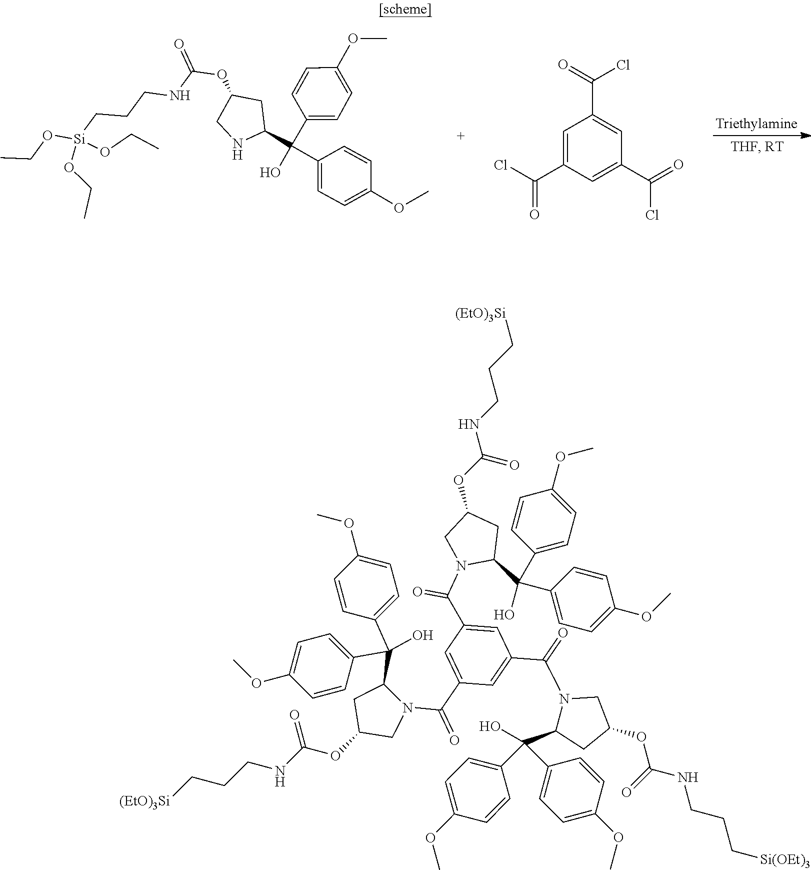

[symbol] Reagent

[symbol]

| | | molecular | | | | molar |
|---|---|---|---|---|---|---|
| item | reactant | weight | amount | | mmole | ratio |
| 1 | product of Step 7 | 576.75 | 0.59 | g | 1.025 | 3.3 |
| 2 | 1,3,5-benzene- | 265.47 | 0.082 | g | 0.310 | 1 |
| | tricarbonyl | | | | | |
| | trichloride, | | | | | |
| | D = 1.487 | | | | | |
| 3 | tetrahydrofuran | — | 20 | mL | — | — |

| | | | | | |
|---|---|---|---|---|---|
| 4 | triethylamine, | 101.19 | 0.93 g/1.3 mL | 9.225 | 9 |
| | D = 0.726 | | | | |

[symbol] Synthesis Steps

[symbol]

The synthesis steps are the same as Step 8 of Example 1.

### Example 8

Recyclable Properties of the Heterogeneous Chiral Catalysts

The heterogeneous chiral catalyst (IV) recovered in Example 4 was tested with the [Selective Reduction Reaction] and [Catalyst Recovery] of Example 4, and the reaction and recovery were repeated three times to verify the recyclable nature of heterogeneous chiral catalyst (IV).

After the reaction was complete, the optical purity and conversion rate of the product were calculated. The results are shown in Table 3.

TABLE 3

| heterogeneous | | | | | R-(+) |
|---|---|---|---|---|---|
| chiral catalyst (IV) | R-(+) | S-(−) | ketone | ee | conversion |
| recovery (1) | 82.3% | 16.7% | 1.0% | 66.2% | 82.3% |
| recovery (2) | 82.6% | 16.3% | 1.1% | 67.1% | 82.6% |
| recovery (3) | 81.9% | 17.2% | 0.9% | 65.3% | 81.9% |

### Example 9

Recyclable Properties of the Heterogeneous Chiral Catalysts

The heterogeneous chiral catalyst (V) recovered in Example 4 was tested with the [Selective Reduction Reaction] and [Catalyst Recovery] of Example 4, and the reaction and recovery were repeated three times to verify the recyclable nature of heterogeneous chiral catalyst (V).

After the reaction was complete, the optical purity and conversion rate of the product were calculated. The results are shown in Table 4.

TABLE 4

| heterogeneous | | | | | R-(+) |
|---|---|---|---|---|---|
| chiral catalyst (V) | R-(+) | S-(−) | ketone | ee | conversion |
| recovery (1) | 87.7% | 11.8% | 0.5% | 76.2% | 87.7% |
| recovery (2) | 88.1% | 11.2% | 0.7% | 77.4% | 88.1% |
| recovery (3) | 87.6% | 11.8% | 0.6% | 76.3% | 87.6% |

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A chiral catalyst, represented by formula (I): 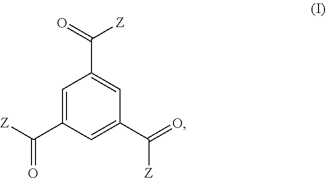
wherein Z═Z₁ or Z₂, and the combination of Z₁ and Z₂ comprises 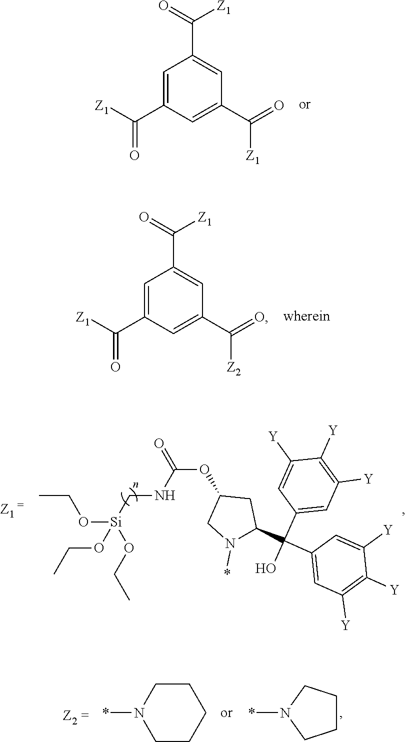
Y independently comprises hydrogen, fluorine, trifluoromethyl, isopropyl, tert-butyl, CₘH₂ₘ₊₁ or OCₘH₂ₘ₊₁, m=1-10, and n=1-10.

2. The chiral catalyst as claimed in claim 1, wherein Y independently comprises hydrogen, CH₃ or OCH₃, and n=3-8.

3. The chiral catalyst as claimed in claim 1, wherein the chiral catalyst is represented by formula (II) or (III): 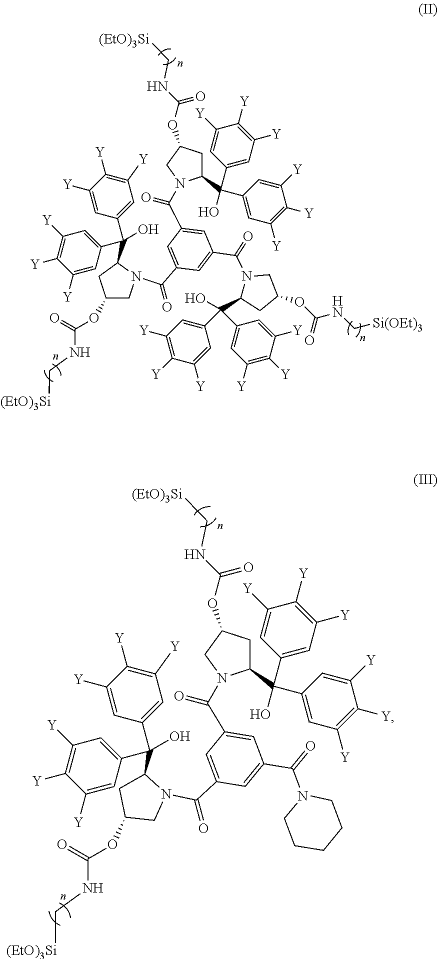
wherein Y independently comprises hydrogen, fluorine, trifluoromethyl, isopropyl, tert-butyl, CₘH₂ₘ₊₁ or OCₘH₂ₘ₊₁, m=1-10, and n=1-10.

4. The chiral catalyst as claimed in claim 3, wherein Y independently comprises hydrogen, CH₃ or OCH₃, and n=3-8.

5. The chiral catalyst as claimed in claim 1, wherein the chiral catalyst comprises

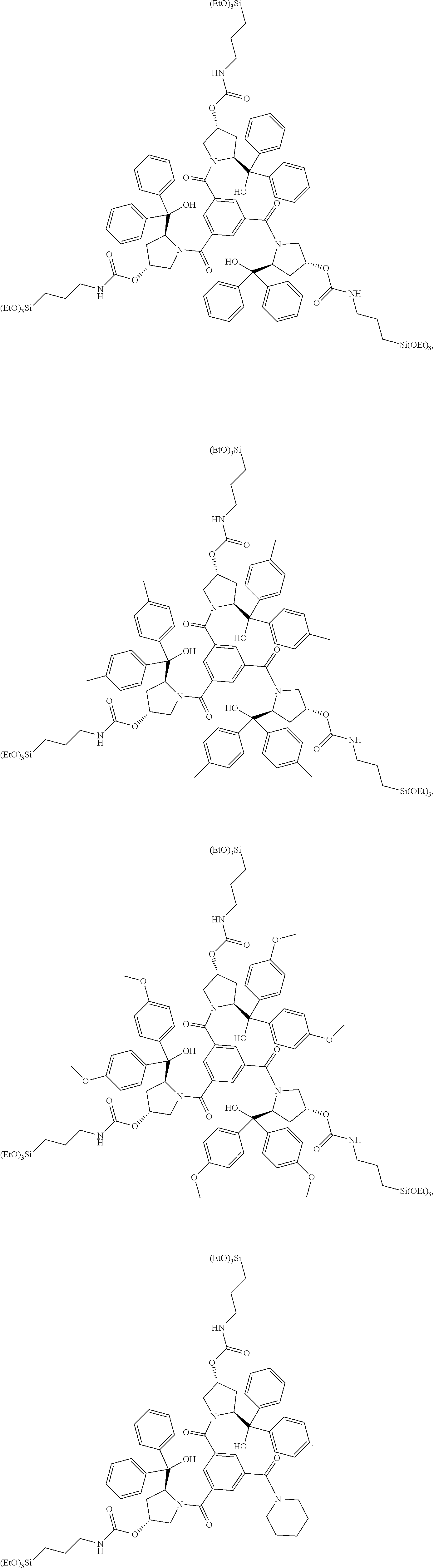

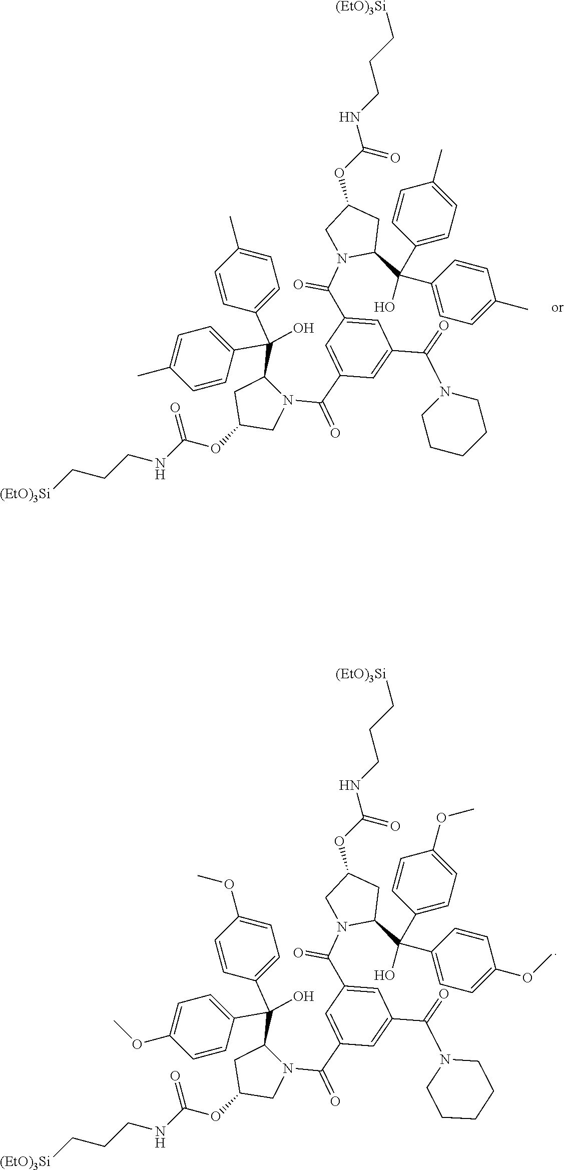

6. A heterogeneous chiral catalyst, comprising:
a chiral catalyst as claimed in claim 1; and
a substrate connected to the chiral catalyst.

7. The heterogeneous chiral catalyst as claimed in claim 6, wherein the heterogeneous chiral catalyst is represented by formula (IV): 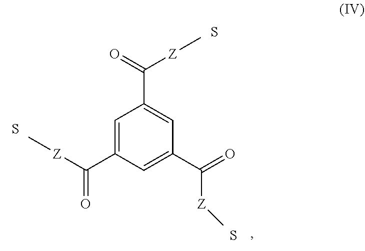
wherein S is the substrate, Z═Z₁ or Z₂, and the combination of Z₁ and Z₂ comprises 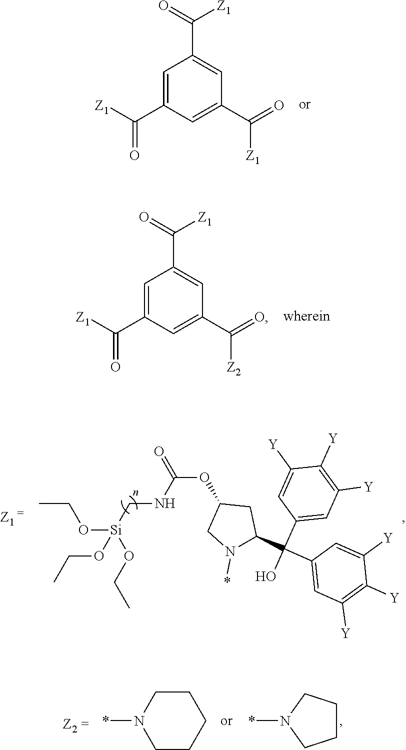
Y independently comprises hydrogen, fluorine, trifluoromethyl, isopropyl, tert-butyl, CₘH₂ₘ₊₁ or OCₘH₂ₘ₊₁, m=1-10, and n=1-10.

8. The heterogeneous chiral catalyst as claimed in claim 7, wherein Y independently comprises hydrogen, CH₃ or OCH₃, and n=3-8.

9. The heterogeneous chiral catalyst as claimed in claim 7, wherein the heterogeneous chiral catalyst is represented by formula (V) or (VI): 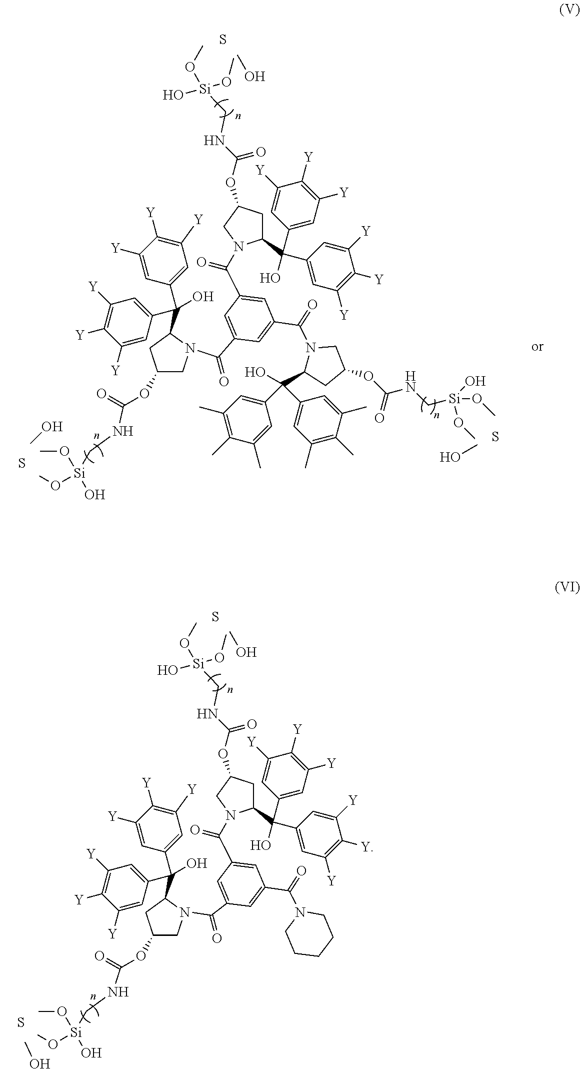
wherein Y independently comprises hydrogen, fluorine, trifluoromethyl, isopropyl, tert-butyl, CₘH₂ₘ₊₁ or OCₘH₂ₘ₊₁, m=1-10, and n=1-10.

10. The heterogeneous chiral catalyst as claimed in claim 9, wherein Y independently comprises hydrogen, CH₃ or OCH₃, and n=3-8.

11. The heterogeneous chiral catalyst as claimed in claim 7, wherein the heterogeneous chiral catalyst comprises

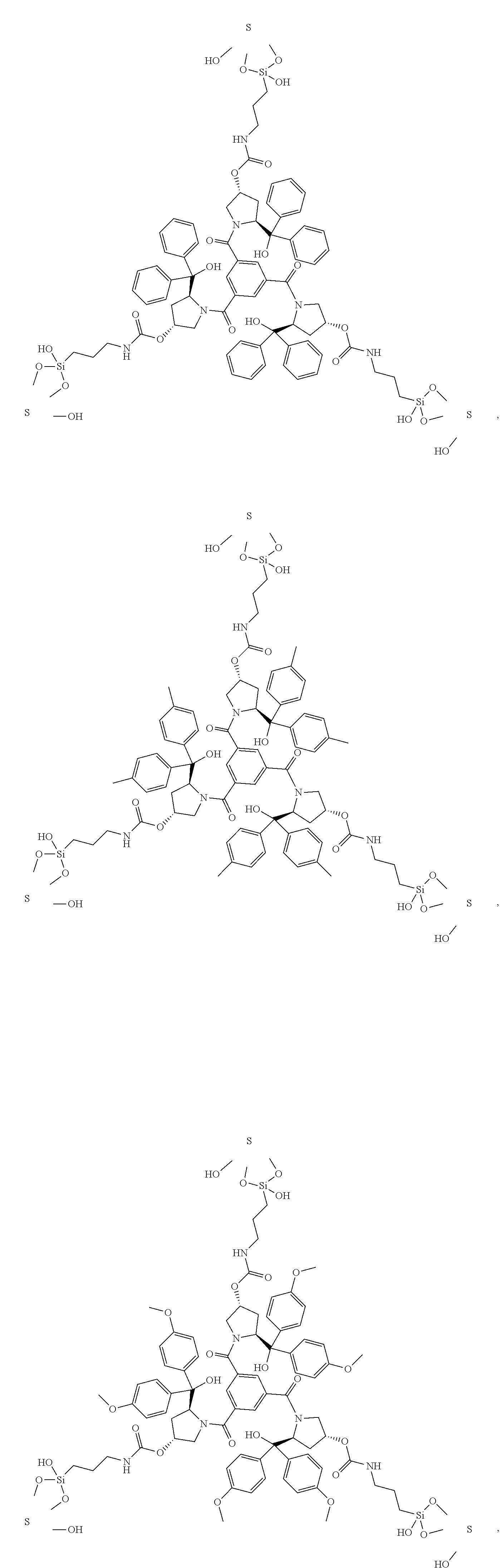

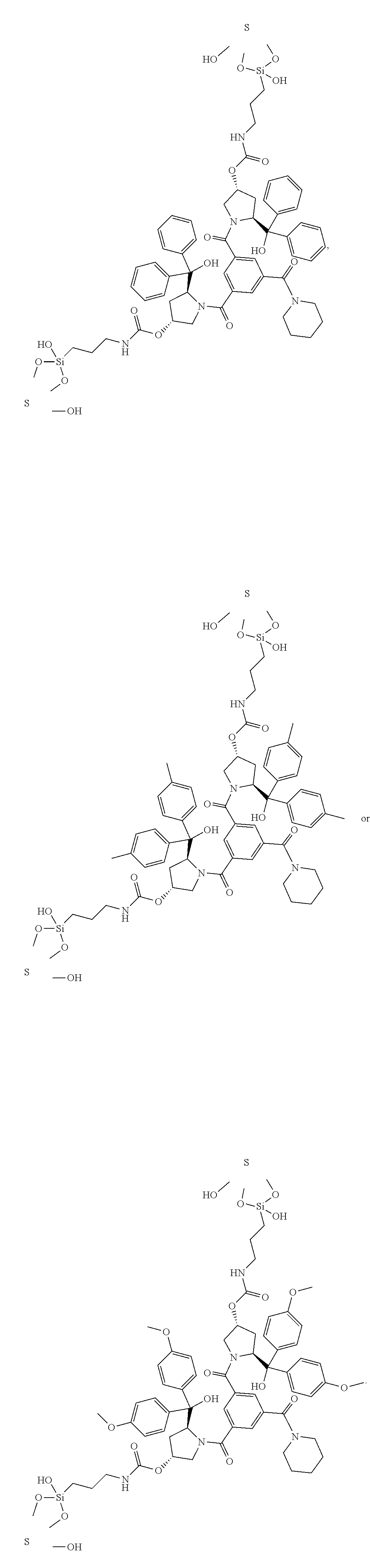

12. The heterogeneous chiral catalyst as claimed in claim 6, wherein the substrate comprises silicon oxide, titanium oxide, iron oxide, zine oxide or aluminum oxide which are modified with a hydroxyl group on a surface of the substrate.

13. The heterogeneous chiral catalyst as claimed in claim 6, wherein the substrate comprises mesoporous material.

14. The heterogeneous chiral catalyst as claimed in claim 13, wherein the substrate has a specific surface area which is in a range from 10 m²/g to 1,000 m²/g.

15. The heterogeneous chiral catalyst as claimed in claim 13, wherein the substrate has a pore size which is in a range from 2 nm to 50 nm.

16. The heterogeneous chiral catalyst as claimed in claim 12, wherein the hydroxyl group of the substrate is connected to the Si(OEt)₃ group of Z₁.

17. The heterogeneous chiral catalyst as claimed in claim 16, wherein a silicon-oxygen bond is formed between the substrate and Z₁.

18. The heterogeneous chiral catalyst as claimed in claim 6, wherein the substrate has an average particle size which is in a range from 5 μm to 500 μm.
